(19) United States
(12) Patent Application Publication
(10) Pub. No.: US 2024/0307277 A1
(43) Pub. Date: Sep. 19, 2024

(54) LOW WATER-CONTENT AND OTHER BROAD-SPECTRUM MINERAL SUNSCREENS

(71) Applicant: LCS Advanced Solutions, LLC, Inglewood, CA (US)

(72) Inventor: Laura C. Singleton, Los Angeles, CA (US)

(21) Appl. No.: 18/661,660

(22) Filed: May 12, 2024

Related U.S. Application Data
(63) Continuation-in-part of application No. 16/566,781, filed on Sep. 10, 2019.

(51) Int. Cl. A61K 8/27; A61K 8/25; A61K 8/29; A61K 8/895; A61K 8/96; A61K 8/9706; A61Q 17/04

(52) U.S. Cl. CPC A61K 8/27; A61K 8/25; A61K 8/29; A61K 8/895; A61K 8/965; A61K 8/9706; A61Q 17/04

(57) ABSTRACT

This disclosure describes broad-spectrum mineral sunscreen compositions (e.g., in the form of a cosmetic compact or stick), which may be untinted or tinted. Such compositions can comprise effective amounts of (a) two photoprotective metal oxides that detectably or significantly block, reflect, refract, or otherwise attenuate ultraviolet radiation, including (i) at least one zinc oxide particle, possibly an uncoated particle, and having an average particle size of greater than about 100 nanometers (ii) and at least one titanium dioxide particle, possibly a coated particle, (b) an amorphous spherical silica and (ii) material comprising light-altering frustules derived from at least one diatomaceous algal species. In some aspects, the algal material is capable of reflecting or absorbing light in a manner distinct from the zinc/titanium dioxide particles. In some aspects, the compositions are characterizable based on the composition's low water content. Such compositions can offer high SPF, water resistance, and non-skin-whitening properties.

### CROSS-REFERENCE TO RELATED APPLICATIONS

This application is a continuation-in-part of U.S. patent application Ser. No. 16/566,781, filed Sep. 10, 2019, and entitled “Mineral, Anhydrous Broad-Spectrum Sunscreen.” This application claims the benefit of priority to and incorporates by reference the entirety of the above-referenced priority application.

### FIELD OF THE INVENTION

This disclosure relates to broad-spectrum, mineral sunscreens that provide protection for human skin across the ultraviolet radiation spectrum (from 280-400 nm).

### BACKGROUND OF THE INVENTION

Mineral (also known as physical) sunscreen filters (most commonly zinc oxide and titanium dioxide) do not raise health concerns of the type associated with chemical (also known as organic) sunscreen filters but, nonetheless, pose challenges both in terms of formulation and aesthetics. Achieving high levels of UVR photoprotection (e.g., a sun protection factor (SPF) of at least 30, which can be described as providing “high protection against sunburn and tanning” under current US Food and Drug Administration (FDA) regulations) with inorganic sunscreen formulations requires high loadings of physical sunscreen filters. Known formulations with high particle loadings can become unstable, and exhibit agglomeration and settling of particles. In addition, physical sunscreen products with high SPF (greater than 30 and, especially, greater than 50) may be difficult to apply (spread, rub-in) and/or may be perceived as lacking cosmetic elegance (in terms of feel and/or appearance). The use of zinc oxide, particularly at higher concentrations, is known to leave a white, pasty residue on the skin.

There has been, and remains, a need for mineral sunscreen formulations that (i) do not contain organic sunscreen actives, (ii) have an SPF of at least 30, preferably of at least 50, (iii) have a critical wavelength of greater than 370 nm, preferably a critical wavelength of greater than 375, (iv) have a high level of water resistance (preferably 80 minutes in accordance with standards set out in Section 352.76 of Title 21 of the U.S. Code of Federal Regulations) and (v) are cosmetically elegant-not oily, not tacky, leave no visible whitening on Fitzpatrick Skin Type I-IV, and are easily applied to the skin (spread). This need is recognized by dermatologists and skin care professionals who are seeking sunscreen formulations suitable for use on their patients/clients with sensitive skin. This need is also recognized by increasingly discerning consumers, especially parents seeking “non-chemical” formulations for use on their children. These needs are met by various sunscreen compositions of the present disclosure.

Surprisingly and unexpectedly, sunscreen formulations of the disclosure have various advantage properties, including, in aspects, exhibiting critical wavelengths of about 375 nm or more, such as 376 nm, or even such as 377 nm or more, as well as addressing some, most, generally all, or all of the above-described shortcomings that are traditionally associated with physical/mineral sunscreens.

### SUMMARY OF THE INVENTION

Disclosed are new compositions that are, among other things, useful as sunscreens. In aspects, the compositions of the invention/disclosure are broad-spectrum mineral sunscreen compositions, which in aspects are presented in the form of compacts and sticks that, in aspects also or alternatively upon application to skin classified as Fitzpatrick Skin Types I-IV (as described in Appendix A), leave no detectable visible deposition (e.g., as evaluated by either a trained evaluator or by other objective measurements and in aspects as confirmed by one or more further evaluations) or a statistically significant visible deposition upon the treated skin as compared to untreated skin (e.g., of a white “pasty” residue) (e.g., as determined by a suitable trial or study conducted by trained persons).

Compositions of this disclosure are characterizable as being at least substantially anhydrous or anhydrous or at least as having low water content. Compositions of the present disclosure may contain, e.g., from about 0.1 wt. % to about 5 wt. % water (e.g., about 0.25-5%, about 0.3-4.8%, about 0.4-4.4%, about 0.5-4.5%, about 0.75-3.75%, about 1-4%, about 1.5-4.5%, or about 2-5% water). Compositions of the present disclosure may be from about 95 wt. % to about 99.5/99.8% wt. % water-free.

Mineral sunscreen compositions of the present disclosure (a) can provide “broad-spectrum protection” from ultraviolet radiation (“UVR”), both UVA and UVB—namely, a sun protection factor (SPF) of at least 30, in some aspects at least 50, and a critical wavelength of at least 370 nm, in aspects at least 375 nm, or in further aspects at least 377 nm (b) can exhibit detectable or significant water resistance, in some embodiments for about 40, about 60, or about 80 minutes or longer, (c) are not visibly whitening on Fitzpatrick Skin Types I-IV, and (d) do not contain an organic sunscreen filter.

Compositions of the invention/disclosure may comprise effective amounts of two or more distinct types of sunscreen-grade metal oxides that block, reflect, refract, or otherwise attenuate ultraviolet radiation in aspects in detectably or significantly different ways. In some respects, compositions of the disclosure can include zinc oxide particles, which, in some aspects, are mostly, generally, substantially entirely, or entirely in uncoated form; in some aspects, are porous, and in some aspects, are capable of being characterized as mesoporous; or in aspects can comprise, mostly comprise, generally consist of, or substantially consist of particles that form aggregates. In aspects, compositions of the disclosure also contain titanium dioxide particles, which in aspects are mostly, generally, substantially, or entirely coated particles (comprising a coating component).

In aspects, compositions comprise at least two siliceous compounds which can comprise, mostly comprise, generally consist of, substantially consist of, or consist entirely of (a) an amorphous silica, which in aspects is mostly, generally, or entirely composed of an amorphous spherical silica, and (b) a component that comprises composition(s) obtained/derived from one or more types of diatomaceous algae (e.g., frustule(s) or compositions comprising frustule(s)), which may, in aspects, reflect UV light, refract UV light, absorb UV light, scatter UV light, block UV light, attenuate UV light, or perform any combination thereof, e.g., for any wavelengths of light between and including UVC (ultraviolet C) to near-IR (near-infrared). In some embodiments, compositions comprise at least two, at least three, or even possibly four or more compositions derived from (“of”) freshwater diatomaceous/light-scattering algae species (e.g., siliceous fragments of freshwater algae species). In aspects, compositions comprise compositions/materials that are derived/obtained from the diatomaceous algae species Fragilaria zeilleri, Gomphonema angustatum, Navicula radiosa, and Cyclotella andancensis. A composition “derived” from a source (e.g., a diatomaceous algae) can be obtained from the source, can be a composition that is otherwise produced but is substantially identical or identical to the source material, or is a material that is modified from the source material by one or more modifications that do not completely, mostly, or significantly diminish the functionality of the source material (and, in aspects, detectably or significantly improve/enhance the intended function of the source material).

In some aspects, compositions also comprise a film-forming component. In aspects, compositions comprise two distinct film-forming polymers. In aspects, such compositions can comprise a film-former component comprising/consisting of copolymers of dimethicone and bis-vinyl dimethicone (e.g., dimethicone (and) bis-vinyl dimethicone/dimethicone copolymer or phenyl trimethicone (and) bis-vinyl dimethicone/dimethicone copolymer) or acrylates—e.g., in aspects, dimethicone (and) acrylates/dimethicone copolymer. In aspects, one or more of these compounds are substituted with materials that are equivalent, generally equivalent, or substantially equivalent in terms of the film-forming properties of such specifically named materials.

In aspects, compositions of this disclosure comprise a wax component, comprising one or more cosmetically suitable waxes. In aspects, compositions comprise at least two waxes having distinct physiochemical properties. In aspects, one or more waxes of a composition function, primarily function, or essentially only function in the composition as a “structuring ingredient” (or “structurer” or “structurant”). In aspects, one or more waxes are capable of and are present in an amount effective to enhance stability, consistency, or both of the compositions detectably or significantly. In aspects, one or more waxes of a composition detectably or significantly increase the solidity, thickness, or viscosity of the composition. In some aspects, some, most, or generally all of the wax components of the compositions are composed of a wax that can be characterized as a mineral wax.

Readers will note that compositions of the invention are variously described as “mineral” compositions, “broad-spectrum” compositions, and the like herein. Readers will recognize that the disclosure of a composition using any such description (e.g., broad-spectrum composition, mineral sunscreen, and the like) is intended to refer to exemplary characteristics of compositions of the disclosure rather than to limit such aspects specifically, and any disclosure of a composition of such a description/type, uncontradicted, implicitly discloses compositions that have any of the other or alternative characteristics of compositions set forth herein (e.g., a composition described as a broad-spectrum sunscreen may also or alternatively be characterizable as a mineral sunscreen, and the like).

Surprisingly and unexpectedly, in aspects, a synergistic combination of an amorphous spherical silica and a mixture of light-altering material (e.g., crystals/frustules) derived from diatomaceous algae, and comprising in aspects materials from one, in aspects two, in additional aspects three, or in further aspects four diatomaceous algae, such as materials from one, some, most, or all of F. zeilleri, G. angustatum, N. radiosa, and C. andancensis, in further combination with particles of zinc oxide and titanium dioxide, particularly in effective amounts, e.g., amounts exemplified herein, can, in embodiments, provide a sun protection factor of at least 30, 40, or 50 (e.g., at least 55, 60, 65, or 70), with a critical wavelength of greater than about 373, 375, or 377, without exhibiting visible skin whitening on Fitzpatrick Skin Types I-IV.

The compositions of the disclosure may also provide one, in some embodiments several, additional/alternative cosmetic skin benefits including (a) reducing the appearance of fine lines/wrinkles, (b) improving skin barrier function (i.e., by reducing the rate/extent of trans-epidermal water loss), (c) reducing oiliness on the skin surface, (d) making the skin feel smoother, more supple and softer, (e) creating the appearance of more even skin tone (reducing dyschromia) and/or (f) imparting “glow” (also described in the art as “radiance” or “brightness”).

### Exemplary Aspects of the Invention

Disclosed here are several different but related exemplary aspects (variations) of the disclosed technology (compositions, methods, etc.).

The following is a non-limiting list of exemplary aspects of the disclosure/invention, which illustrates embodiments of the disclosure/invention in an alternative summary form to aid readers in quickly understanding the overall scope of the invention.

Similar to patent claims, listed aspects described in the paragraphs of this section may make reference to (i.e., depend on/from) one or more other paragraphs. Readers will understand that such references mean that the features/characteristics or steps of such referenced aspects are incorporated into/combined with the referring aspect. E.g., if an aspect in a paragraph (e.g., a paragraph indicated by text at the end of the paragraph as aspect 2) refers to another aspect by one or more aspect numbers (e.g., aspect 1 or “any one of aspects 1-3”), it will be understood to include the elements, steps, or characteristics of such referenced aspects (e.g., aspect 1) in addition to those of the aspect in which the reference is made (e.g., if aspect 2 refers to aspect 1, it provides a description of a composition, method, system, device, etc., including the features of both aspect 1 and aspect 2).

Lists of aspects describing specific exemplary embodiments of the invention are sometimes employed to aid the reader in understanding the disclosure. Such aspects can, within them, reference other exemplary aspects, either individually or as groups of aspects (e.g., via reference to a range within a list of numbered aspects when such aspects are provided as a numbered list). Reference to ranges of aspects should be interpreted as referencing all such aspects individually, each as unique embodiments of the invention, and in combination with one another as unique embodiment(s) of the invention, according to the presentation provided of such aspects unless such an aspect within such a referenced range is either contradictory or non-sensical. If contradicted, reference to the contradictory aspect should be excluded.

It is intended that these listed exemplary aspects begin with the first listed aspect (ASPECT 1) and thereafter be numbered sequentially and incrementally by inclusion of a reference placed near or at the end of the listed aspect (ASPECT 2, ASPECT, 3, etc.). In case of a missing aspect reference or repeated aspect reference, the order of placement of the actual recited aspect in the list that is associated with the repeated aspect reference or missing aspect reference will control (e.g., if there is an unlabeled aspect located between a first aspect labeled ASPECT 1 and a third aspect labeled ASPECT 2, the unlabeled aspect should be treated as ASPECT 2, and the aspect labeled as ASPECT 2 treated as ASPECT 3, etc.), and all numbering in the list (including all references to aspects in the list) be interpreted as accordingly modified (e.g., if the fourth aspect in such list was labeled as ASPECT 3, it should be interpreted as being labeled as ASPECT 4, and if such aspect referred to “any one or both of aspect 1 or aspect 2,” it should be read as referring to “any one or more of aspects 1-3”). Similarly, if an aspect is misnumbered (e.g., by a number in the sequence being skipped or otherwise missing), readers will similarly construe this list of aspects according to the order of placement of the recited aspects, over the numerical references. Further, if one or more of the listed exemplary aspects of the invention in this section fails to reference any other aspects of the invention, such aspect, uncontradicted, should be interpreted as applying to or as capable of being incorporated into, any one or more other exemplary aspect(s) provided in this section.

In a first aspect, this disclosure provides a composition that comprises an effective amount of one or more cosmetically suitable zinc oxide particles that reflects, refracts, or otherwise attenuates ultraviolet radiation; an effective amount of one or more cosmetically suitable titanium dioxide particles that reflects, refracts, or otherwise attenuates ultraviolet radiation; an effective amount of at least two cosmetically suitable siliceous compounds comprising an amorphous spherical silica composition and a composition comprising material derived from at least one diatomaceous algal species comprising a porous silica frustule structure; an effective amount of dimethicone and acrylates/dimethicone copolymer, bis-vinyl dimethicone/dimethicone copolymer, or a combination thereof; an amount of one or more cosmetically suitable waxes that is sufficient to promote the maintenance of a solid or semisolid structure at room temperature; and comprises only (no more than) about 0.25% to about 5% water; wherein the composition exhibits an SPF of at least 30 (e.g., at least 40, 50, 60, or 70), has a critical wavelength of at least 370 nm (e.g., 373 or 375 nm or more), and is water resistant for at least about 80 minutes, and wherein the composition does not contain any detectable amount of an organic sunscreen ingredient. ASPECT 1.

An effective amount in this regard can mean, e.g., an amount that is effective in imparting the intended/described function of the ingredient (e.g., the attenuation of UV radiation, the enhancement of SPF, the improvement of structure in terms of viscosity, solidity, etc., or imparting water-resistance or non-whitening effects).

In an aspect, the disclosure provides a composition according to ASPECT 1, wherein the weight ratio of the zinc oxide particles to the titanium dioxide particles is about 1:4 to about 3:1. ASPECT 2.

The disclosure further provides a composition wherein the weight ratio of the zinc oxide particles to the titanium dioxide particles is about 1:55 to about 1:1.5. ASPECT 3.

The disclosure further provides a composition according to any one or more of ASPECTS 1-3, wherein most of the zinc oxide particles of the composition are mesoporous particles, and a detectable amount of the zinc oxide particles are contained in zinc oxide particle aggregates. ASPECT 4.

The disclosure further provides a composition according to any one or more of ASPECTS 1-4, wherein the weight ratio of the diatomaceous algae composition to the amorphous spherical silica composition is about 1:20 to about 1:50. ASPECT 5.

The disclosure further provides a composition according to ASPECT 5, wherein the composition comprises only about 0.5% to about 5% water. ASPECT 6.

The disclosure further provides a composition according to any one or more of ASPECTS 1-6, wherein the diatomaceous algae composition comprises material derived from two distinct diatomaceous algal species. ASPECT 7.

The disclosure further provides a composition according to any one or more of ASPECTS 1-7, wherein the one or more cosmetically suitable waxes comprise a mineral wax, wherein the weight ratio of the mineral wax to the total of the one or more cosmetically suitable waxes is about 1:1.5 to about 1:4. ASPECT 8.

The disclosure further provides a composition according to any one or more of ASPECTS 1-8, wherein the composition comprises an amount of a mineral wax that detectably or significantly enhances the viscosity of the composition, solidity of the composition, or both, wherein the mineral wax is mostly composed of ozokerite. ASPECT 9.

The disclosure further provides a composition according to any one or more of ASPECTS 7-9, wherein the composition comprises only about 1% to about 5% water. ASPECT 10.

The disclosure further provides a composition according to any one or more of ASPECTS 1-10, wherein the composition further comprises an effective amount of at least one ingredient SPF booster compound, which significantly increases the SPF of the composition but does provide a significant amount of SPF protection in the absence of the zinc oxide particles and titanium dioxide particles. ASPECT 11.

The disclosure further provides a composition according to ASPECT 11, wherein the weight ratio of the SPF booster to the at least two siliceous compounds to the combination of the zinc oxide particles and titanium dioxide particles is about 20:3:20 to about 30:5:30. ASPECT 12.

The disclosure also provides compositions that contains less than an effective amount of any organic sunscreen active pharmaceutical ingredient and comprises an effective amount of one or more cosmetically suitable zinc oxide particles, that reflects, refracts, or otherwise attenuates ultraviolet radiation; an effective amount of one or more cosmetically suitable titanium dioxide particles, that reflects, refracts, or otherwise attenuates ultraviolet radiation; an effective amount of at least two cosmetically suitable siliceous compounds, wherein at least one of the at least two cosmetically suitable siliceous compounds is an amorphous spherical silica and at least one of the at least two siliceous compounds comprises frustules derived from at least one diatomaceous algal species and comprising a photonic and porous silica crystal structure; an effective amount of dimethicone and acrylates/dimethicone copolymer, bis-vinyl dimethicone/dimethicone copolymer, or a combination thereof; an amount of one or more cosmetically suitable waxes sufficient to promote the composition having a solid or semisolid structure at room temperature; and about 0.25% to about 5% water, a composition water, by weight; wherein the composition exhibits an SPF of at least 30, has a critical wavelength of at least 370 nm, is water resistant for at least about 80 minutes according to a test method of Section 352.76 of Title 21 of the United States Code of Federal Regulations as follows (a) applying the broad-spectrum mineral sunscreen composition, (b) after an applicable waiting period, performing 20 minutes of moderate activity in water; (c) waiting for a 20-minute rest period, (d) performing 20 minutes of moderate activity in water; and (e) waiting for a 20-minute rest period without wiping the broad-spectrum mineral sunscreen composition with a towel, and wherein the composition does not cause visible whitening on the skin of a person having a Fitzpatrick Skin Type of I-IV. ASPECT 13.

The disclosure further provides a composition according to ASPECT 13, wherein the composition comprises about 0.5% to about 5% composition water by weight, such as about 1% to about 5% composition water by weight, such as about 1.5% to about 5% composition water by weight, such as 2-5%, 1.5-4.5%, 2-4%, 1.25-4.25%, 1.25-3.75%, 1.75-3.5%, 1.75-4.5%, 1.75-4.25%, or about 2.5% to about 5% composition water by weight. ASPECT 14.

As described herein, uncontradicted, the term water means free (unassociated/unbound) water and does not include, for example, structural water contained in hydrates, crystalline structures, and the like.

The disclosure further provides a composition according to any one or more of ASPECTS 13 or 14, wherein the zinc oxide particles have (a) an average particle size less than about 0.8 microns based on the particle number size, and (b) the zinc oxide particles are mostly, generally only, or entirely mesoporous. ASPECT 15.

The disclosure further provides a composition according to any one or more of ASPECTS 13-15, wherein most, generally all, or all of the diatom frustules of the composition have a silica content of at least about 75 wt. %. ASPECT 16.

The disclosure further provides a composition according to any one or more of ASPECTS 13-16, wherein the weight ratio of (i) the frustules to (ii) the amorphous spherical silica is from about 1:20 to about 1:50. ASPECT 17.

The disclosure further provides a composition according to any one or more of ASPECTS 13-17, wherein the frustules are derived from at least two diatomaceous algal species. ASPECT 18.

The disclosure further provides a composition according to any one or more of ASPECTS 13-18, wherein the weight ratio of the zinc oxide particles to the titanium dioxide particles is about 1:4 to about 3:1. ASPECT 19.

The disclosure further provides a composition according to any one or more of ASPECTS 13-19, wherein the weight ratio of the at least two siliceous compounds to the zinc oxide particles and titanium dioxide particles is about 1:55 to about 1:1.5. ASPECT 20.

The disclosure further provides a composition according to any one or more of ASPECTS 13-20, wherein the composition further comprises at least one ingredient which significantly increases in vivo SPF but doesn't alone contribute significantly to the SPF of the composition (an SPF booster). ASPECT 21.

The disclosure further provides a composition according to any one or more of ASPECTS 13-21, wherein the weight ratio of the SPF booster to the at least two siliceous compounds to the zinc oxide particles and titanium dioxide particles is about 20:3:20 to about 30:5:30. ASPECT 22.

The disclosure further provides a composition according to any one or more of ASPECTS 13-22, wherein the one or more cosmetically suitable waxes comprise a mineral wax, wherein the weight ratio of the mineral wax to the total of the one or more cosmetically suitable waxes is about 1:1.5 to 1:4. ASPECT 23.

The disclosure further provides a composition according to ASPECT 23, wherein the mineral wax comprises, mostly comprises, generally consists of, or consists of ozokerite. ASPECT 24.

The disclosure further provides a composition according to any one or more of ASPECTS 13-24 (or ASPECTS 1-12), wherein the composition does not comprise an emulsion. ASPECT 25.

The disclosure further provides a broad-spectrum mineral sunscreen composition comprising: two sunscreen-grade metal oxide particles that block, reflect, refract, or otherwise attenuate ultraviolet radiation comprising (a) an effective amount, mostly comprising, generally consisting of, consisting essentially of, or consisting of (i) zinc oxide particles that are not coated and have a mesoporous structure (and in aspects an average particle size of fewer than 0.8 microns, e.g., 0.1-0.8 microns), and titanium dioxide particles that (a) have an average particle size of greater than about 100 nanometers, and (b) are coated with a suitable coating (e.g., triethoxycaprilylsilane, silica, hydrogen dimethicone, stearic acid, isostearic acid, polyhydroxystearic acid, alumina, aluminum hydroxide, caprylic/capric triglyceride, or a combination of any or all thereof); (b) at least two siliceous compounds comprising an effective amount, mostly comprising, generally consisting of, essentially consisting of, or consisting of an amorphous spherical silica, and at least two diatomaceous algal species comprising a photonic and porous silica crystal structure, the algal species selected from the group consisting of Fragilaria zeilleri, Gomphomena angustatum, Navicula radiosa, and Cyclotella andancensis; (c) an effective amount of a film forming component, in aspects comprising, mostly comprising, generally consisting of, or consisting of (i) dimethicone and acrylates/dimethicone copolymer, and (ii) bis-vinyl dimethicone/dimethicone copolymer; (d) at least two physiochemically distinct structuring waxes (e.g., in which at least one of the at least two waxes is a natural wax comprising a fixed oil expressed from the kernel of a plant, unsaponifiables within plant kernel oil, or both, and at least one of the at least two waxes is a mineral wax or a synthetic wax); and (e) about (i.e., only about/not more than) 0.5% to about 5% water by weight, wherein the mineral sunscreen composition has an SPF of at least 30, and wherein the mineral sunscreen composition is free of an effective amount of any organic sunscreen active pharmaceutical ingredient (or is free of any detectable amount of an organic sunscreen active ingredient). ASPECT 26.

The disclosure further provides a composition according to ASPECT 26, wherein the composition further comprises pigmentary-grade titanium dioxide particles having an average particle size of up to about 30 microns. ASPECT 27.

The disclosure further provides a composition according to ASPECT 26 or ASPECT 27, wherein the composition further comprises at least two silicone compounds comprising dimethicone/vinyl dimethicone crosspolymer and a silicone fluid selected from the group consisting of dimethicone, simethicone, and phenyl trimethicone. ASPECT 28.

The disclosure further provides a composition according to any one or more of ASPECTS 26-28, wherein the composition further comprises two or more compounds from among butyloctyl salicylate, octyldodecyl neopentanoate, and polyhydroxystearic acid. ASPECT 29.

The disclosure further provides a composition according to any one or more of ASPECTS 26-29, the composition further comprises at least one compound which significantly provides a cosmetic skin benefit selected from the group consisting of (i) Bisabolol, (ii) Zingiber officinale (Ginger) root extract, (iii) Himanthalia elongata extract, (iv) cetylhydroxyproline palmitamide, (v) cerium oxide, (vi) tocopheryl acetate, and (vii) ubiquinone. ASPECT 30.

The disclosure further provides a composition according to any one or more of ASPECTS 26-30, wherein the composition is water resistant for at least 80 minutes according to a test method of Section 352.76 of Title 21 of the United States Code of Federal Regulations as follows: (a) applying the broad-spectrum mineral sunscreen composition; (b) after an applicable waiting period, performing 20 minutes of moderate activity in water; (c) waiting for a 20-minute rest period; (d) performing 20 minutes of moderate activity in water; and (e) waiting for a 20-minute rest period without wiping the broad-spectrum mineral sunscreen composition with a towel. ASPECT 31.

The disclosure further provides a composition according to any one or more of ASPECTS 26-31, wherein the composition does not cause visible whitening on the skin of a person having a Fitzpatrick Skin Type of I-IV. ASPECT 32.

The disclosure further provides a composition according to any one or more of ASPECTS 26-32, wherein the composition has a critical wavelength of at least 373, 374, 375, or 376 nm. ASPECT 33.

The disclosure further provides a composition according to any one or more of ASPECTS 26-33, wherein application of the composition to skin detectably or significantly diminishes the appearance of fine lines and wrinkles. ASPECT 34.

The disclosure further provides a method of protecting the skin of a user/treated person against UV radiation comprising applying an effective amount of a composition according to any one or more of ASPECTS 1-34 to the skin of the treated person/user. ASPECT 35. A user in this respect can be any person that self-administers a composition of the invention, and a treated person can be any person that has a composition of the invention applied with the assistance of another person. Readers will understand that each such term, when used herein, provides implicit support for a corresponding disclosure in which the term is substituted with the other term.

### DETAILED DESCRIPTION OF THE INVENTION

### Guidance for Interpretation and Description of Select Terms/Concepts

This section offers guidelines for reading this disclosure.

The intended audience for this disclosure (“readers”) are persons having at least ordinary skill(s) in the practice of technologies discussed or used herein. Readers may also be called “skilled persons,” and such technologies and related publicly available prior knowledge are collectively referred to as “the art.” Terms such as “understood,” “known,” and “ordinary meaning” refer to the general knowledge of skilled persons.

The term “uncontradicted” means not contradicted by this disclosure, logic, or plausibility based on knowledge of skilled persons.

Disclosed here are several different but related exemplary aspects (variations) of the systems/units/devices, or compositions, methods, and other subject matter of this disclosure (also referred to as, e.g., “cases,” “facets,” “respects,” or “embodiments”). All aspects as described individually and as can be arrived at by any combination of such individual aspects can be used to characterize a claimed/claimable invention of this disclosure. Thus, any reference to “aspects” (e.g., “according to aspects” or “in an aspect”) will be understood as referring to according to any and all of the other suitable aspects described herein. In this respect, the breadth and scope of the disclosure and inventions provided by this disclosure should not be limited by any exemplary aspect(s)/embodiment(s). No language in this disclosure should be construed as indicating any element/step is essential to any aspect, group of aspects, or category of aspects unless such requirement is explicitly stated. Uncontradicted, any aspect(s) described in any part of this disclosure can be combined with any other aspect(s) in any other part. Uncontradicted, the term “invention” can be used to refer to an inventive aspect or combination of aspects of the disclosure or to possible groups/types of aspects. In passages where the term is used very generally readers can interpret any usage of “invention” as synonymous with “disclosure.” No usage of the term “invention” is intended to require any certain feature to be included or to be present in a claimed system/device/composition or method associated with this disclosure.

Disclosure herein is directed to things (e.g., compositions, devices, or systems) as well as method(s) comprising the use of such things and other step(s)/procedure(s). Disclosure of aspects directed to or defined by things should be construed as simultaneously providing support for the use of such things in method(s)/procedure(s). Similarly, disclosure of method(s)/procedure(s) wherein system(s)/element(s) are provided should be construed as simultaneously providing support for corresponding things that can carry out the step(s) of such method(s)/procedure(s) based on relevant elements of this disclosure, the knowledge of the art, or both.

Uncontradicted, any elements, steps, components, or features of aspects and all variations thereof, etc., are within the scope of the disclosure and can be combined to characterize claimed/claimable inventions.

Uncontradicted, all technical/scientific terms used here should be read, at least in one aspect, to have the same meanings as commonly understood by skilled persons, regardless of any narrower examples or descriptions provided here (including any term introduced initially in quotations). However, readers will also recognize that some aspects can characterized by the inclusion of elements, steps, features, characteristics, etc., associated with specific descriptions provided here and that such specific disclosures represent distinct embodiments of the disclosure apart from the corresponding aspect that is provided by interpreting the disclosure using any broader commonly used terminology or concept. Uncontradicted, the disclosure of any aspect using known terms, which terms are narrowed by example or otherwise, implicitly discloses one or more related aspects in which the applicable terms are alternatively interpreted using the broadest reasonable interpretation of skilled persons.

Uncontradicted, the term “or” means “and/or” here, regardless of any explicit inclusion of the actual phrase “and/or” (e.g., phrases such as “A, B, or C” and “A, B, and/or C” each simultaneously discloses aspects including (1) all of A, B, and C; (2) A and C; (3) A and B; (4) B and C; (5) only A; (6) only B; and (7) only C (and also support sub-groupings, such as “A or B,” “A or C,” etc.).

Uncontradicted, the term “also” means “also or alternatively.” Uncontradicted, the terms “here” & “herein” mean “in this disclosure.” The term “i.a.” (“ia” or “ia”) means “inter alia” or “(possibly) among other things.” “Also known as” can be abbreviated “aka” or “AKA.” (and can mean is otherwise referred to, even if the relationship between the terms is not well known). The term “elsewhere” means “elsewhere herein.”

For conciseness, symbols may be used herein. E.g., “&” may be used to represent the word “and,” & “˜” to represent the word “about.” Symbols such as < and > are given their ordinary meaning (e.g., “≤” means “less than or equal to” & “≥” means “greater than or equal to”). A slash “/” between terms here can represent “or” (“A/B” means “A or B”) or identify synonyms of an element, depending on context. The inclusion of “(s)” after an element or a step indicates that ≥1 of such an element is present, step performed, and the like. E.g., the term “element(s)” refers to both 1 element and ≥2 elements, with the understanding that each thereof is an independent aspect of the disclosure. Uncontradicted, where an element is only provided in standard plural form (e.g., “compositions” as opposed to composition(s)), the reader should interpret such disclosure as encompassing a single composition as if presented as “composition(s).”

Use of the abbreviation “etc.” (or “et cetera”) in association with a list of elements/steps means any or all suitable combinations of the recited elements/steps or any known equivalents of such recited elements/steps for achieving the function(s) of such elements/steps known in the art. Readers should interpret phrases like “and the like” similarly.

Uncontradicted, terms such as “and combinations,” “or combinations,” and “combinations thereof,” etc., regarding listed elements/steps, means any or all possible/suitable combinations of the associated elements/steps. Thus, e.g., uncontradicted, a phrase like “combination of any thereof” refers to any or all combinations.

Aspects may be described as suitable for use(s) disclosed herein. Uncontradicted, terms such as “suitable” or “suitability” mean acceptable, appropriate, or, in aspects practical for performing a particular function/achieving particular state(s)/outcome(s), and typically means effective, practical, and non-deleterious/harmful in the context the term is used. E.g., uncontradicted, the term “suitable” means appropriate, acceptable, or in contexts sufficient, or providing at least generally or substantially all an intended function (of the element or overall whole of the aspect), without causing or imparting significant negative/detrimental impact. Uncontradicted, each element of the disclosure is suitable for its intended use. In some aspects, suitability can be demonstrated through scientific studies and to a degree of significance through suitable tests/measures such as scientific tests, well-controlled and adequate studies (e.g., pre-clinical or clinical studies), adequately powered consumer testing, judgment by relevant experts, consumer approval/surveys, or regulatory authority review, and as established by, e.g., scientific measures, commercial success, expert opinions (e.g., medical opinions), and the like. Components, elements, and steps described as “suitable” should be interpreted in a similar manner.

Steps, elements, devices, components, and the like also or alternatively can be described as “effective.” Uncontradicted, any disclosed element is to be construed as being effective for its intended purpose and present in an effective amount, and any step performed is to be understood as being performed/applied effectively, such as in an effective amount or an effective number of times, etc. Uncontradicted, efficacy can be judged by evaluating the element(s) ability to perform or contribute to the described function(s) or characteristic(s) associated with the component, device, step, etc., the overall aspect, or both. Efficacy in this respect when applied to effects in organisms, such as people, can mean, and, uncontradicted, should be interpreted to implicitly disclose efficacy that can be measured (1) in a treated subject/user, (2) in a majority of subjects in a population, (3) in a statistically significant number of subjects in a population, (4) generally all subjects in a population, (5) substantially all subjects in a population, or (6) in a statistically significant number of or more of a typical or average subject of the class of subjects treated.

Constituents herein are typically present in “effective” quantity(ies), e.g., length(s), ratios, and the like. Uncontradicted, any described class/type of, e.g., component is understood to be present in the associated system or method in an effective state/quantity, e.g., length(s), ratio(s), and the like.

In the context of this disclosure, “effective” uncontradicted refers to an amount, concentration, etc., that results in an effective intended result associated with the associated ingredient, component, composition, or compound, etc. For example, for a sunscreen ingredient, an effective amount is an amount that results in an effective sunscreen effect (e.g., a significant sun protection effect in a significant number of treated persons/users in an adequately powered population of such treated persons/users).

Uncontradicted, heading(s) and subheadings used here are included for convenience and do not limit the scope of any aspect(s). Uncontradicted, aspect(s), step(s), or element(s) described under one heading can apply to other aspect(s) or step(s)/element(s).

Uncontradicted, ranges of values here represent each value falling within a range within an order of magnitude of the smallest endpoint of the range without having to write each value of the range explicitly. E.g., a recited range of 1-2 implicitly discloses each of 1.0, 1.1, 1.2, . . . 1.9, and 2.0, and 10-100 implicitly discloses each of 10, 11, 12, . . . 98, 99, and 100). Uncontradicted, all ranges include the range's endpoints, regardless of how a range is described. E.g., “between 1-5” includes 1 and 5 in addition to 2, 3, and 4 (and all numbers between such numbers within an order of magnitude of such endpoints, e.g., 1.0, 1.1, . . . 4.9, and 5.0). For the avoidance of doubt, any number within a range, regardless of the order of magnitude of the number, is covered by the range (e.g., a range of 2-20 covers 18.593). Uncontradicted, readers will understand that any two values in a range provided as a list herein can be combined as endpoints to form a range defining a more particular aspect of the disclosure (e.g., if a list of values 1, 2, 3, 4, and 5 of element X is provided, readers will understand that the disclosure implicitly discloses an aspect comprising 2-4 X, 3-5 X, and 1-3 X, etc.

Terms of approximation (e.g., “about,” “˜,” or “approximately”) can be used here (1) to refer to a set of related values or (2) where a precise value is difficult to define (e.g., due to limits of measurement). Uncontradicted, all exact values provided here simultaneously/implicitly disclose corresponding approximate values and vice versa (e.g., disclosure of “about 10” provides explicit support for the use of 10 exactly in such aspect/description). Ranges described with approximate value(s) include all values encompassed by each approximate endpoint, regardless of presentation (e.g., “about 10-20” has the same meaning as “about 10-about 20”). The scope of value(s) encompassed by an approximate term typically depends on the context of the disclosure, criticality or operability, statistical significance, understanding of the art, etc. In the absence of guidance here or in the art for an element, terms such as “about” when used in connection with an element should be interpreted as ±10% of the indicated value(s) and implicitly disclosing ±5%, ±2%, ±1%, and ±0.5%. In a similar manner, uncontradicted, a range of ratios explicitly provided in this disclosure implicitly describes ranges of ratios contained within the disclosed ratios, wherein the end points of each such implicitly disclosed ratio are within an order of magnitude of the explicitly disclosed ratios. For example, where the disclosure provides an explicit disclosure of “about 1:10 to about 1:100,” uncontradicted, readers will understand that such disclosure provides implicit support for sub-ranges thereof, such as about 1:10 to 1:50, about 1:50 to about 1:100, and about 1:25 to about 1:75, among others.

This disclosure includes aspects associated with particular characteristics, such as amounts or lengths of components (or ranges thereof), In cases, several such characteristics of varying scope may be provided. Readers will understand that each such characteristic can be associated with particular properties that distinguish such aspects from other aspects, and, accordingly, each such range can be viewed as critical to a particular aspect of the disclosure, even if the associated results, properties, functions, etc., associated with such aspects are not directly communicated in association with such characteristics.

Lists of aspects, elements, steps, and features are sometimes employed for conciseness. Unless indicated, each member of each list should be viewed as an independent aspect. Each aspect defined by any individual member of a list can have and often will have, nonobvious properties vis-a-vis aspects characterized by other members of the list.

Uncontradicted, the terms “a” and “an” and “the” and similar referents encompass both the singular and the plural form of the referenced element, step, or aspect. Uncontradicted, any aspect disclosed herein with an element or step expressed in the singular provides implicit support for a corresponding embodiment in which the element(s)/step(s) are present in the plural (two or more), and vice versa (in other words, disclosure of an element/step implicitly discloses the corresponding use of such/similar elements/steps and vice versa). Hence, e.g., a passage regarding an aspect including X step supports a corresponding aspect including several X steps. Uncontradicted, any mixed use of a referent such as “a” in respect of one element/step or characteristic and “one or more of” with respect to another element/step or characteristic in a paragraph, sentence, aspect, or claim, does not change the meaning of such referents. Thus, for example, if a paragraph describes a composition comprising “an X” and “one or more Ys,” the paragraph should be understood as providing disclosure of “one or more Xs” and “one or more Ys.”

Uncontradicted, terms such as “significant” and “significantly” mean results/characteristics that are statistically significant using ≥1 appropriate test(s)/trial(s) in the given context (e.g., p≤0.05/0.01). Terms such as “detectable” mean measurably present/different using known detection tools/techniques. The acronym “DOS” (or “DoS”) means “detectable(ly) or significant(ly).” The term “measurably” means at a measurable level. The term detectable provides implicit disclosure for aspects that are “measurable,” and the term “measurable” implicitly supports aspects where the measured or measurable element is “detectable.” Compositions/ingredients described herein as being associated with a function are to be understood to implicitly provide such a function in an at least detectable, at least significant, or at least effective amount. For example, an ingredient described as a “soft-focus powder” will be understood to impart, when present in an effective amount, a detectable improvement in, a significant improvement in, or an effective improvement in a soft-focus appearance (through skin smoothing, reduction of the appearance of pores, etc.). Any disclosure of an ingredient, component, compound, etc., herein that is not associated with a specified amount will be understood to be present in an effective amount.

Uncontradicted, for any value here that is not accompanied by a unit of measurement (e.g., a length of 5, a force of 3, a concentration of 2, or a distance of 4), readers will understand that either any previously provided unit for the same element/step or the same type of element/step will apply, or, in cases where no such disclosure exists, the unit of measure most used in association with such an element/step in the art (given the relevant context) applies.

Uncontradicted, the terms “including,” “containing,” “comprising,” and “having” mean “including, but not limited to,” or “including, without limitation.” Uncontradicted, use of terms such as comprising and including regarding elements/steps means including any detectable number or amount of an element or including any detectable performance of a step/number of steps (with or without other elements/steps). Uncontradicted, “a” means one or more, even when terms such as “one or more” or “at least one” are used in association with the referent “a.”

For conciseness, description of an aspect “comprising” or “including” an element, with respect to a collection/whole (e.g., a system, device, or composition), implicitly provides support for any detectable amount/number or ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the whole/collection being made up of the element, or essentially all of the whole/collection being made up of the element (i.e., that the collection consists essentially of the referenced element). Similarly, a method described as including a step with respect to an effect/outcome implicitly provides support for the referenced step, providing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the effect/outcome, representing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the steps/effort performed, or both. Explicit listing of percentages of elements in connection with particular aspects does not limit or contradict such implicit disclosure. Uncontradicted, readers should interpret terms such as “essentially all” or “essentially” consistent with the concept of “consisting essentially of.” The basic and novel properties/advantages of compositions that are associated with aspects “consisting of” elements are provided throughout this disclosure (e.g., water-resistance, high SPF protection, high critical wavelength, and non-whitening properties of compositions).

Uncontradicted, terms such as “comprising” when used in connection with a step of a method provide implicit support for performing the step once, ≥2 times, or until an associated function/effect is achieved.

Uncontradicted, any disclosure of an object or method (e.g., composition, device, or system) “comprising” or “including” element(s) provides implicit support for an alternative corresponding aspect that is characterized by the object consisting of that element or “consisting essentially of” that element (excluding anything that would “materially affect” the “basic and novel characteristic(s)” of the subject matter of the disclosure). Uncontradicted, any specific use of phrases such as “consists of” and “consists essentially of” herein does not modify this construction principle.

Readers will understand the “basic and novel characteristic(s)” of the subject matter of this disclosure, and the scope of what constitutes a “material effect” (or “material effect”) of such “basic and novel characteristics” will vary with the specific applicable aspect of the subject matter at issue. Uncontradicted, the basic and novel characteristics of any aspect of the disclosure include the specific recited and associated elements of an aspect and exclude any other element that significantly detracts from the intended function(s) of the recited elements, that introduce significant new functions that are unrelated to the intended function(s), that significantly reduce the performance of the function(s), or that significantly negatively change other characteristics of performing such function(s) (e.g., by increasing the cost of performing the functions in energy, money, or both). Uncontradicted, the basic and novel characteristics also include at least significantly retaining the suitability, effectiveness, or both of an explicitly described/referenced aspect, thing, or method. Accordingly, a material effect can be an effect that reduces, diminishes, eliminates, counteracts, cancels, or prevents one or more of such functions in one or more respects (e.g., delaying onset, reducing scope, reducing duration, reducing output, reducing the level of applicability, reducing effect, or combinations thereof). In an aspect, a material effect is one that changes such functions by making such functions impractical, difficult to obtain, or materially more expensive or otherwise costly in terms of inputs. From this and the other guidance provided herein, readers can understand the scope of an aspect that is defined by consisting essentially of a collection of elements.

Uncontradicted, the term “one” means a single type, single iteration/copy/thing, of a recited element or step, or both, which will be clear from the context of the relevant disclosure. For example, the referent “one” used with respect to a component of a composition/article or system can refer to one type of element (which may be present in numerous copies, as in the case of an ingredient in a composition) one unit of the element, or both. Similarly, “one” component, a “single” component, or the “only component” of a system typically means 1 type of element (which may be present in numerous copies), 1 instance/unit of the element, or both. Further, “one” step of a method typically means performing one type of action (step), one iteration of a step, or both. Uncontradicted, a disclosure of “one” element provides support for both, but uncontradicted, any claim to any “one” element means one type of such an element (e.g., a type of component of a composition/system/article).

Uncontradicted, the term “some” means ≥2 copies/instances or ≥5% (e.g., ≥7.5%, ≥12.5%, ≥17.5%, ≥27.5%, or ≥37.5%) of a listed collection/whole is or is made up of an element. Regarding methods, some means ≥5% of an effect, effort, or both is made up of or is attributable to a step (e.g., as in “some of the method is performed by step Y”) or indicates a step is performed ≥2 times (e.g., as in “step X is repeated some number of times”). Terms such as “predominately,” “most,” or “mostly” (and “primarily” when not used to refer to an order of events or “mainly”) means >50% (e.g., mostly comprises, predominately includes, etc., mean >50%) (e.g., a system that mostly includes element X is composed of >50% of element X). The term “generally” means ≥75% (e.g., generally consists of, generally associated with, generally comprises, etc., means ≥75%) (e.g., a method that generally consists of step X means that 75% of the effort or effect of the method is attributable to step X). “Substantially” or “nearly” means ≥95% (e.g., nearly all, substantially consists of, etc., mean ≥95%) (e.g., a collection that nearly entirely is made up of element X means that at least 95% of the elements in the collection are element X). Terms such as “generally free” of an element or “generally lacking” an element mean comprising ≤25˜% of an element, and terms such as “substantially free” of an element mean comprising ≤˜5% of an element. Uncontradicted, any aspect described as “generally comprising” or “generally consisting” of an element implicitly discloses an element that “substantially comprises” the element. The same principle applies to any disclosure where an aspect is described as being “generally free” of an element.

Uncontradicted, any disclosure that an element or step is lacking from a thing or method provides implicit support for the element or step being substantially absent, essentially absent, generally absent, or mostly absent/not performed.

Uncontradicted, any aspect described with respect to an optionally present element(s)/step(s) also provides implicit support for corresponding aspect(s) in which one, some, most, generally all, nearly all, essentially all, or all of such element(s) are lacking/step(s) not performed, in respect of the relevant aspect. Readers will understand that uncontradicted, any element, feature, step, or characteristic of any aspect of the invention recited herein as being present in an aspect also implicitly provides support for the element, feature, step, or characteristic as being excluded from a corresponding/similar aspect of the invention implicitly disclosed by the explicit positive disclosure. For example, if the disclosure states that in an aspect, object A comprises element B, readers will understand that a corresponding aspect also is implicitly disclosed where object A lacks element B.

Uncontradicted, changes to tense or presentation of terms (e.g., using “comprises predominately” in place of “predominately comprises”) do not change the meaning of the corresponding term/phrase.

Uncontradicted, all methods provided here can be performed in any suitable order regardless of presentation (e.g., a method comprising steps A, B, and C, can be performed in the order C, B, and A; B and A and C simultaneously, etc.). Uncontradicted, elements of a composition can be assembled in any suitable manner by any suitable method. In general, any methods and materials similar or equivalent to those described here can be used in the practice of embodiments in at least the broadest version of the relevant aspect. Uncontradicted, the use of ordinal numbers such as “first,” “second,” “third,” and so on is primarily, though not exclusively, intended to distinguish respective elements rather than to limit the disclosure to a particular order of those elements, importance, or configuration.

Uncontradicted, any elements, steps, components, or features of aspects and all variations thereof, etc., are within the scope of the invention and broadest version of any aspect.

Any elements associated with a function can be alternatively described as “means for” performing a function in a composition/device/system or a “step for” performing a part of a method, and parts of this disclosure refer to “equivalents,” which means known equivalents known in the art for achieving a referenced function associated with disclosed mean(s)/step(s). However, no element of this disclosure or claim should be interpreted as limited to a “means-plus-function” or “step-plus-function” construction unless such intent is clearly indicated by the use of the terms “means for” or “step for.” Terms such as “configured to” or “adapted to” do not indicate “means-plus-function” interpretation, but, rather, describe element(s)/step(s) configured to, designed to, selected to, or adapted to achieve a certain performance, characteristic, property, or the like using teachings provided here or in the art

[symbol]

As used herein, the word “exemplary” means “serving as an example, instance, or illustration.” The following detailed description is merely exemplary in nature and is not intended to limit application and uses. Any embodiment described herein as “exemplary” is not necessarily to be construed as preferred or advantageous over other embodiments. Except where explicitly indicated or clearly indicated by context, “improved” herein means “increased.” In aspects, “improved” means “reduced,” such as with respect to the toxicity of a composition. Uncontradicted, terms such as “enhanced,” “improved,” and the like are used synonymously.

All references (e.g., publications, patent applications, and patents) cited herein are hereby incorporated by reference as if each reference were individually and specifically indicated to be incorporated by reference and set forth in its entirety herein. Uncontradicted, any suitable principles, methods, or elements of such references (collectively “teachings”) can be combined with or adapted to aspects. However, citation/incorporation of patent documents is limited to the technical disclosure thereof and does not reflect any view regarding the validity, patentability, etc., thereof. In the event of any conflict between this disclosure and the teachings of such documents, the content of this disclosure controls regarding aspects of the invention. Numerous references are cited here to concisely incorporate known information and aid skilled persons in putting aspects into practice. While efforts have been made to include the most relevant references for such purposes, readers will understand that not every aspect of every cited reference will apply to every aspect of the invention.

### Description of Terms/Concepts Specific to Sunscreen Technology in This Disclosure

The term “emulsion” usually refers to a two-or three-phase system in which water (“W”) is dispersed in oil (“O”) and/or silicone (“S”); or silicone/oil is dispersed in water. Emulsions are commonly abbreviated “X”/“Y” where “X” is the internal, discontinuous phase that is dispersed in an external continuous phase “Y.” For example, emulsions are described in the art as, e.g., W/O (water-in-oil); O/W (oil-in-water); W/Si; Si/W; and W/O/W). Compositions of this disclosure can be in aspects characterized by the lack of any emulsion or by being essentially free or substantially free of any emulsion.

The broad-spectrum photoprotective compositions of the present disclosure can be characterized by the absence of any detectable, significant, or effective amount of any one or more organic sunscreens/sunscreen actives. Accordingly, in describing and claiming such compositions as “consisting essentially of (a) two sunscreen-grade metal oxides it is meant that: zinc oxide particles (in aspects uncoated particles) and titanium dioxide (in aspects coated particles) are present as the active ingredients of the sunscreen composition and there is nothing contained in the composition that would significantly change the nature of the sunscreen component of the composition. In aspects, such compositions can comprise compatible other metal oxides that block, reflect, refract, or otherwise attenuate visible, infrared, or ultraviolet radiation, including cerium oxide, aluminum oxide, and iron oxides, typically in concentrations/amounts such that the zinc and titanium oxide particles continue to provide most, generally all, substantially all, or all of the sunscreen effects of the composition, as such additional metal oxide components do not change the essential characteristic of this aspect of the invention.

Terms such as “organic sunscreen” will be understood to mean an active ingredient that characterized as a sunscreen active ingredient by the U.S. Food and Drug Administration (“FDA”) at the time of or prior to the time of this application other than the metal oxide particle active ingredients described herein—e.g., namely, avobenzone; cinoxate; dioxybenzone; ecamsule; homosalate; menthyl anthranilate; octocrylene; octyl methoxycinnamate; octyl salicylate; oxybenzone; p-aminobenzoic acid; padimate o; phenylbenzimidazole sulfonic acid; sulisobenzone; and trolamine salicylate. Organic Sunscreen active ingredient(s) herein are in aspects alternatively referred to as “Organic Sunscreen Active,” “Chemical Sunscreen,” or “Organic Sunscreen Active Pharmaceutical Ingredient(s)” and the like. Organic Sunscreen in aspects also can include active ingredients approved by regulatory agencies outside the United States at the time of or prior to the time of this application but not approved by the FDA at or prior to the time of this application, including, 4-methylbenzylidene camphor, amiloxate, benzophenone-9, Mexoryl® XL, Neo Heliopan® AP, Parsol® SLX, Tinosorb® A2B, Tinosorb® M, Tinosorb® S, Uvasorb® HEB, Uvinul® A Plus, and Uvinul® T 150.

“Sunscreen-Grade Metal Oxide” means particle(s) that can, in aspects physically block ultraviolet, visible and/or infrared light, and thereby can provide some degree of protection to the skin, and in aspects reduce environmentally-caused damage which can manifest as fine lines, wrinkles, uneven pigmentation (dyschromia), loss of elasticity or firmness, increased dryness, reduced skin moisture, loss of softness and suppleness, or any combination thereof.

“Pigmentary-Grade Titanium Dioxide” means particle(s) of TiO₂, which in aspects have an average particle size of up to about 30 microns. In aspects, pigmentary-grade titanium dioxide is a particle that is not recognized as a sunscreen metal oxide by FDA or another regulatory agency.

Particle size may be determined using techniques and equipment well-known to a person having ordinary skill in the art, including, for example, Mastersizer® 3000 and Mastersizer® 300E particle sizing devices available from Malvern Panalytical Inc. (Westborough, MA) as well as using LS 13 320 MW Particle Size Analysis and Multisizer 4e Particle Size Analysis from Beckman Coulter Inc. (Allendale, NJ), among others.

The term “broad-spectrum” is generally understood in the art. In aspects, terms like “broad-spectrum” refer to a level of protection from UVR provided by wearing a sunscreen that has a minimum critical wavelength of 370 nm and a sun protection factor (SPF) value of about 15 or higher.

Similarly, the term “critical wavelength” is known in the art. In aspects, “critical wavelength” is the wavelength for which the section under the integrated optical density curve starting at 290 nm is equal to 90 percent of the integrated section between 290 nm to 400 nm. Standards and test methods for determining “broad-spectrum” protection from UVR are set out in the final Sunscreen Monograph promulgated by the FDA on Jun. 17, 2011. See “Labeling and Effectiveness Testing; Sunscreen Drug Products for Over-the-Counter Human Use” published at Volume 76 of the Federal Register starting at page 35620, the disclosure of which is incorporated herein by reference.

Unless otherwise indicated, percentages, parts, and ratios are to be understood as based upon the total weight of the composition and may be expressed in aspects as “wt. %.”

A “cosmetic/aesthetic benefit” is an improvement in appearance, for example, a visible reduction in appearance of fine lines/wrinkles, an improvement in skin barrier function (by reducing the rate/extent of trans-epidermal water loss), skin that feels smoother/more supple/softer, skin that appears to have more even tone (reduced dyschromia), skin that appears to have “glow” or “radiance” (also described in the art as “brightness”), or any combination thereof. As described elsewhere, such measurements/outcomes can be determined or evaluated by any suitable method and level (e.g., by having a detectable or significant effect). Uncontradicted, any composition disclosed herein will be understood as being capable of providing cosmetic/aesthetic benefit to a user/treated person (e.g., to a population of users/treated persons, such as an adequately powered population of users/treated persons).

The disclosure herein provides composition(s), e.g., broad-spectrum sunscreen composition(s) comprising a plurality of ingredient(s) or component(s). Disclosed ingredient(s) and component(s) are typically cosmetically suitable, e.g., safe for contact with human skin. In aspects, ingredient(s) and component(s) are compatible with a keratinous substrate such as human skin such that application to human skin in amount(s) described herein does not cause damage, pain, or other adverse effect(s) preventing safe and effective use of composition(s) in which the ingredient(s)/component(s) are used.

As noted elsewhere, the disclosures of all US-granted patents and US pre-grant patent publications in this disclosure are incorporated, in pertinent, by reference and the teachings thereof can be combined with the teachings of this disclosure so long as such teachings are uncontradicted by this disclosure.

To assist readers in understanding the invention both combinations of elements/steps of things/methods provided by this disclosure and individual elements/steps thereof may be described in this section of this disclosure.

Despite the inclusion of any passages focused on specific elements/steps, readers will understand that any aspect, facet, embodiment, or other description of a particular step(s) or element(s) can be applied to any general description of the compositions/methods of the invention, or any other recited element(s)/step(s) thereof, which are provided in any part of this disclosure.

The invention provides, in aspects, compositions, which can be variously characterized as broad spectrum/mineral sunscreen compositions, that comprise at least an effective amount of one or more cosmetically suitable zinc oxide particles that reflects, refracts, or otherwise attenuates ultraviolet radiation; an at least effective amount of one or more cosmetically suitable titanium dioxide particles that reflects, refracts, or otherwise attenuates ultraviolet radiation; an at least effective amount of a siliceous compound component that modulates the light reflection/scattering, refraction, absorption, or attenuation of the compositions comprising at least an effective amount of a diatomaceous algal material derived from one or more species (and which, in aspects, comprises or consists of at least two cosmetically suitable siliceous compounds comprising an amorphous spherical silica composition and a composition comprising material derived from at least one diatomaceous algal species comprising a porous silica frustule structure); an at least effective amount of dimethicone and acrylates/dimethicone copolymer, bis-vinyl dimethicone/dimethicone copolymer, or a combination thereof; an amount of one or more cosmetically suitable waxes that is sufficient to promote the maintenance of a solid or semisolid structure at room temperature; and comprises only/no more than about 0.25% to about 5% water (e.g., only ˜0.3-4.5%, only ˜0.3-4.2%, only ˜0.3-3.9%, only ˜0.3-3.6%; only about 0.5-3.5%, only about 0.5-4%, only about 1-4%, only about 1-4.5%, only ˜1-3.5%, only ˜2-4.5%, or only ˜2-4% water) wherein the composition exhibits an SPF of at least 30 (e.g., at least 40, at least 45, at least 50, at least 60, or about 70 or more), has a critical wavelength of at least 370 nm (e.g., at least 372 nm, at least 374 nm, or at least about 375 nm), and is water resistant for at least about 40 minutes, at least about 60 minutes, or at least about 80 minutes (e.g., 10-100, 10-90, 10-80, 20-80, 20-90, 30-90, 30-80, or ˜40-80 minutes), and wherein the composition does not contain any effective amount or, in aspects, even any detectable amount of an organic sunscreen ingredient.

Readers will understand that terms such as “effective amount” are to be construed as meaning a suitable amount that is “at least an effective amount.” even where the phrase “an at least effective amount” is used in parts of this disclosure. In other words, aspects described with respect to the inclusion of an effective amount of one or more ingredients will be understood as referring to compositions in which an amount of such ingredient(s) are present in at least effective amounts up to the maximally suitable amount of such ingredient(s). Effective amounts can be determined based on the guidance provided herein and knowledge of the art in view of the specific functions ascribed to the components of compositions described herein.

To better exemplify these and other types of compositions provided by this disclosure, the following sections will focus on these and other ingredients that can be included in the compositions of the disclosure.

### Sunscreen-Grade Metal Oxide Particles

Broad-spectrum photoprotective compositions of the present disclosure in aspects contain, zinc oxide (ZnO) and titanium dioxide (TiO₂), or both types of “Photoprotective Metal Oxides”.

TiO₂ can be amorphous or crystalline in the rutile and/or anatase form. In aspects, 95% or more of the TiO₂ is in the rutile form, with less than about 5% of the TiO₂ in an anatase form or some other non-rutile form, or any suitable and effective combination of anatase or non-rutile form.

In aspects, a composition comprises pigmentary-grade titanium dioxide particles. In aspects, some, most, generally all, substantially all, essentially all, or all of pigmentary-grade titanium dioxide particles of a composition are sunscreen-grade metal oxide particles. In aspects, some, most, generally all, substantially all, essentially all, or all of pigmentary-grade titanium dioxide particles are photoprotective metal oxides. In aspects, pigmentary-grade titanium dioxide particles are distinguishable from active sunscreen titanium dioxide particles in terms of the ability to provide a sunscreen effect.

ZnO and TiO₂ particles in certain embodiments can be coated. Coating may be done by methods known in the art. By way of a non-limiting example, starting with an aqueous pigment particle suspension, metal salts can be added in a dissolved form as so-called precursor compounds. Alkaline or acid substances are then used to set the pH value of the suspension in such a way that the precursor compounds are precipitated in the form of oxides, hydroxides, etc. Methods for modifying and hydrophobizing the surface of TiO₂ and ZnO are further disclosed, for example, in U.S. Pat. Nos. 5,565,591 and 5,486,631, both now expired.

In aspects, some, most, generally all, substantially all, essentially all, or all of the particles of one, some, most, generally all, substantially all, or all of the types of particles of a composition are characterizable as being porous particles, e.g., as being mesoporous particles. In aspects, some, most, generally all, substantially all, essentially all, or all of the particles of a type (e.g., zinc oxide particles, irregular zinc particles, or other metal oxide particles) have pores that somewhat, mostly, generally only, substantially only, essentially only, or only have a diameter (or maximum dimension) of about 2-about 50 nm. In aspects, all, nearly all, substantially all, essentially all, generally all, most, or some of the particle pores in some, most, generally all, substantially all, or all of the particles are not characterizable as micropores (under 2 nm in size/diameter) or macropores (e.g., over 50 nm in size/diameter).

In aspects, examples of materials that can be used to coat ZnO and TiO₂ particles are silica [SiO₂], alumina [Al₂O₃], aluminum hydroxide [Al(OH)₃·xH₂O] (which may introduce an amount of water into a composition which depends on the mass of aluminum hydroxide used and the value of X as will be understood in the art), aluminum stearate [CH₃(CH₂)₁₆COOAl(OH)₂], triethoxycaprylylsilane (CH₃CH₂O)₃Si(CH₂)₇CH₃], stearic acid, caprylic/capric triglyceride, lecithin, and methicone.

Additionally, TiO₂ may have a coating approved by the Scientific Committee on Consumer Safety of the European Commission in document SCCS/1580/16.

Photoprotective metal oxides, such as but not limited to TiO₂ and ZnO, may also be a mixture of C₁₂ to C₃₀ fatty alcohols and C₆ to C₁₂ aliphatic acids, as described in U.S. Pat. No. 9,517,190.

TiO₂ particles that can be useful in broad-spectrum mineral sunscreen compositions of the present disclosure may be commercially available in a mixture that is a “binary combination”—namely, TiO₂ and a second ingredient selected from the group of: dimethicone; lecithin; isopropyl titanium triisostearate; methicone; polymethyl methacrylate; polyphosphorylcholine glycol acrylate; silica; simethicone; stearic acid; and triethoxycaprylylsilane.

In certain embodiments, the broad-spectrum mineral sunscreen composition contains a mixture of TiO₂ and lecithin. Readers will understand from this disclosure that the inclusion of additional components in such a particle does not change the fact that the particle is a titanium dioxide particle. In this regard, metal oxide particles can, uncontradicted, contain additional compounds or components that are suitable for combination with the metal oxide component(s) of the particles (e.g., coatings as exemplified elsewhere herein).

TiO₂ useful in broad-spectrum mineral sunscreen compositions of the present disclosure may also be part of a tripartite combination (i.e., TiO₂ and a second ingredient, and a third ingredient).

In certain embodiments, TiO₂ and alumina (as a second ingredient) are combined with a third ingredient selected from the group of: glycerin; jojoba esters; methicone; silica; and stearic acid in particles of a composition.

In other embodiments, TiO₂ and aluminum hydroxide (as a second ingredient) are combined with a third ingredient selected from the group of: hydrogen dimethicone; isostearic acid; and stearic acid.

In further embodiments, TiO₂ and silica (as a second ingredient) are combined with a third ingredient selected from the group of: Helianthus annuus (Sunflower) Seed Oil; dimethicone; stearic acid; jojoba esters; lauroyl lysine; sodium polyacrylate; and triethoxycaprylylsilane.

In still further embodiments, TiO₂ and polyhydroxystearic acid (as a second ingredient) are combined with a third ingredient selected from the group of: bisabolol; squalane; and jojoba esters.

TiO₂ and caprylic/capric triglyceride may in aspects be combined with alumina, and polyhydroxystearic acid and one of: aluminum stearate; methicone; stearic acid; silica TiO₂ and caprylic/capric triglyceride may be combined with aluminum hydroxide, polyhydroxystearic acid and/or stearic acid.

TiO₂ may in aspects be combined with C₁₂₋₁₅ alkyl benzoate, polyhydroxystearic acid, and, optionally alumina, in further combination with one of: methicone; cyclomethicone; aluminum stearate; stearic acid, and silica.

TiO₂ may in further aspects also be combined with C₁₂₋₁₅ alkyl benzoate in further combination with: (i) Argania spinosa kernel oil (and) alumina (and) methicone (and) tocopheryl acetate; (ii) Dimethicone (and) polyhydroxystearic acid (and) silica; (iii) Polyglyceryl-3 polyricinoleate (and) silica (and) stearic acid (and) aminopropyl-triethoxysilane; (iv) stearic acid (and) aluminum hydroxide (and) polyhydroxystearic acid.

TiO₂ may also in certain aspects be combined with aluminum hydroxide in further combination with: (i) Acrylates copolymer (and) hydrated silica (and) algin; (ii) Butyloctyl salicylate (and) isostearic acid (and) C₁₂₋₁₅ alkyl benzoate (and) stearic acid; (iii) C₁₂₋₁₅ alkyl benzoate (and) stearic acid (and) polyhydroxystearic acid; (iv) Caprylic/Capric triglyceride (and) stearic acid in further combination with (a) sorbitan olivate or (b) Polyhydroxystearic acid; (v) Hydrogen dimethicone, alone or in combination with hydrogen dimethicone; (vi) Hydrated silica (and) polyphosphorylcholine glycol acrylate; (vii) Stearic acid or isostearic acid; (viii) Polydimethylsiloxyethyl hexyl dimethicone (and) PEG-9 polydimethylsiloxyethyl dimethicone; (ix) Polyglyceryl-4 isostearate (and) cetyl PEG/PPG-10/1 dimethicone (and) hexyl laurate (and) isostearic acid; (x) Simmondsia chinensis (Jojoba) Seed Oil (and) isostearic acid (and) polyhydroxystearic acid.

Non-limiting examples of “coated” TiO₂ suitable for use in broad-spectrum mineral sunscreen compositions of the present disclosure include the following: (i) Sunsil® Tin50 from Sunjin Chemical Co. Ltd.: TiO₂ coated with silica, with a ratio of silica to TiO₂ of about 55:45; (ii) Titanium dioxide (at least 78%; typically about 83%) coated with aluminum hydroxide (about 9%) (and) stearic acid (about 8%), available from Tayca Corp. (Osaka, Japan) under the tradename MT-100TV; (iii) Titanium dioxide (74%) coated with silica (11%), aluminum hydroxide (9%), and alginic acid (5%), available from Tayca Corp. as MT100-AQ; (iv) Titanium dioxide (75-82%) coated with silica (13-20%) available from Merck KgaA/EMD Chemicals (Darmstadt, Germany) under the tradename Eusolex® T-AVO; (v) SiClone® TD-150 (from Presperse Corp., Somerset, New Jersey) about 40% titanium dioxide with an inner coating of aluminum hydroxide and an outer coating of isostearic acid.

TiO₂ in certain embodiments may be used in broad-spectrum mineral sunscreen compositions of the present disclosure in one of the following combinations: (a) Boron nitride (and) dimethicone (and) isododecane (and) ethylene/vacopolymer; (b) Butylene glycol (and) caprylyl glycol (and) oleth-10 (and) phenoxyethanol (and) polysorbate 60 (and) silica; (c) Butyloctyl salicylate (and) polyhydroxystearic acid (and) dimethicone (and) hydrogen dimethicone; (d) Caprylic/Capric triglyceride (and) stearic acid (and) isostearic acid (and) polyhydroxystearic acid (and) polyglyceryl-3 polyricinoleate (and) lecithin; (e) Cyclomethicone (and) Bis-PEG/PPG-14/14 dimethicone (and) aluminum stearate; (f) Cyclopentasiloxane (and) dimethicone (and) PEG-10 dimethicone (and) silica; (g) Cyclopentasiloxane (and) PEG-10 dimethicone (and) methicone; (h) Ethylene/Acrylic Acid copolymer (and) aluminum stearate; (i) Ethylhexyl palmitate (and) polyhydroxystearic acid (and) silica; G) Glycerin (and) sodium polyacrylate (and) tetrasodium EDTA (and) silica (and) sodium polyphosphate; (k) Hydrogenated polydecene (and) polyhydroxystearic Acid (and) one of: (i) Dimethicone; (ii) Stearic acid; or (iii) Triethoxycaprylylsilane; isododecane (and) polyhydroxystearic acid (and) methicone; (m) Isohexadecane (and) triethylhexanoin (and) aluminum stearate (and) polyhydroxystearic acid; (n) Isononyl isononanoate (and) methicone (and) polyhydroxystearic acid; (o) Isopropyl myristate (and) polyhydroxystearic acid (and) silica; (p) Isopropyl titanium triisostearate (and) triethoxysilylethyl polydimethylsiloxyethyl dimethicone; (q) Methyl trimethicone (and) hydrogen dimethicone (and) lauryl PEG-9 polydimethylsiloxyethyl dimethicone; (r) Methyl trimethicone (and) PEG-10 dimethicone (and) methicone; (s) Mica (and) dimethicone (and) isododecane (and) ethylene/va copolymer (and) stearic acid; (t) Octyldodecyl myristate (and) alumina (and) polyhydroxystearic acid (and) methicone; (u) Phenyl trimethicone (and) hexyl laurate (and) stearic acid (and) polyhydroxystearic acid; (v) Polyglyceryl-2 caprate (and) sucrose stearate (and) Simmondsia chinensis (Jojoba) Seed Oil (and) stearic acid (and) glyceryl caprylate (and) squalane; (w) Simmondsia chinensis (Jojoba) Seed Oil (and) aluminum hydroxide (and) polyhydroxystearic acid (and) one of isostearic acid or jojoba esters; (x) Acrylates copolymer (and) hydrated silica (and) one of (i) Algin (and) aluminum hydroxide or (ii) Polyphosphorylcholine glycol acrylate; (y) Butyloctyl salicylate (and) Aluminum hydroxide (and) isostearic acid (and) C₁₂₋₁₅ alkyl benzoate (and) stearic acid; (z) C₁₂₋₁₅ alkyl benzoate (and) polyglyceryl-2 dipolyhydroxystearate (and) silica (and) dimethicone; (aa) Caprylic/Capric triglyceride (and) sorbitan olivate (and) stearic acid (and) aluminum hydroxide; (bb) Caprylyl methicone (and) cyclopentasiloxane (and) C₁₂₋₁₅ alkyl benzoate (and) alumina (and) polyhydroxystearic acid (and) triethoxysilylethyl polydimethylsiloxyethyl hexyl dimethicone (and) PEG-9 polydimethylsiloxyethyl dimethicone; (cc) Hydrated silica (and) hydrogen dimethicone and (a) Aluminum hydroxide (or) hydrogen dimethicone; (dd) Isododecane (and) alumina (and) methicone (and) polyhydroxystearic acid; (ee) Isododecane (and) polyglyceryl-4 isostearate (and) cetyl PEG/PPG-10/1 dimethicone(and) hexyl laurate (and) aluminum hydroxide (and) isostearic acid; (ff) Isononyl isononanoate (and) polyhydroxystearic acid (and) hydrogen dimethicone (and) dimethicone.

In one embodiment of the disclosure, titanium dioxide suitable for use in broad-spectrum mineral sunscreen compositions of the present disclosure is non-whitening when applied to the skin and has an average particle size greater than about 100 nm (i.e., “non-nano”). One non-limiting example of a non-nano TiO₂ is the product currently sold under the brand name Tinoply E50C (manufactured by Chemland, Co., Ltd., Seoul, Korea), a mixture of caprylic/capric triglyceride, titanium dioxide, alumina, stearic acid, and polyhydroxystearic Acid (all products described herein with a brand name are in reference to those products as available at the time this disclosure is initially made or at the last time such products were commercially available if before the date of this disclosure). More particularly, in Tinoply E50C (a) Caprylic/Capric triglyceride is present at a concentration of 47.0±2.5%, (b) Titanium dioxide is present at a concentration of 39.0±2.5%, (c) Alumina is present at a concentration of 6.0±2.5%, (d) Stearic acid is present at a concentration 5.0±2.5%, and (e) Polyhydroxystearic acid is present at a concentration of 3.0±0.5%.

In another embodiment of the disclosure, titanium dioxide suitable for use in broad-spectrum mineral sunscreen compositions of the present disclosure may be Tinoply E30C—a mixture of caprylic/capric triglyceride, titanium dioxide, silica, polyhydroxystearic acid, and methicone (also manufactured by Chemland, Co., Ltd.) in which (a) Caprylic/Capric triglyceride is present at a concentration of about 68.2±2.5%, (b) Titanium dioxide is present at a concentration of at least about 24%, (c) Silica is present at a concentration of 4.5±2.5%, (d) Polyhydroxystearic acid is present at a concentration 1.8±0.5%, and (e) Methicone at a concentration of 1.5±0.5%.

In certain embodiments of the disclosure, a broad-spectrum mineral sunscreen does not include alumina.

Uncoated zinc oxide particles that can be used in certain exemplary compositions of the present disclosure are commercially available from numerous suppliers, including under the tradename Z-Cote® (BASF Care Creations, Florham Park, NJ). Examples of other suitable ZnO are disclosed, for example, in U.S. Pat. No. 8,545,891.

In certain embodiments of the disclosure, ZnO is non-whitening when applied to the skin and also has an average particle size greater than about 100 nm (i.e., is a “non-nano” particle). Such materials are sometimes described in trade literature as “transparent.” Historically, non-whitening, non-nano ZnO materials were marketed under the tradename ZinClear™ formerly sold by Antaria Limited (Welshpool, Australia). See US Pre-Grant Patent Application Publications 2010/0310871 and 2010/0316582, both abandoned.

In certain embodiments of the disclosure, compositions of the present disclosure contain ZnO particles having an average particle size of greater than 100 nanometers. One such ZnO is ARGA-SUN ZnO CLR-P from Argan Co. (Northridge, California). According to Technical Data Sheets, ARGA-SUN ZnO CLR-P has a highly porous structure that may, in aspects, be “infiltrated” (e.g., filled) with an excipient, and an average particle size distribution of less than about 800 nanometers (measured using static laser scattering). Without desiring to be bound to a particular theory, it has been hypothesized that because the particles may be not only be highly porous but also mesoporous and have a refractive index close to the excipient, a “significant increase in transparency” is achieved, that is in certain aspects detectably or significantly less likely to leave a white residue on the skin of one or more users. Other examples of suitable ZnO particles which may be used in certain embodiments, alone or in combination with other ZnO particles include ARGA-SUN ZnO 700 SiP6 and ARGA-SUN ZnO CLR-P-TE.

In aspects, ZnO particles suitable for use in composition(s) herein include mesoporous zinc oxide aggregates (MZOAs) having an average particle size of 0.8 microns (800 nm) that produce detectably or significantly more visibly transparent mineral composition(s) than traditionally utilized zinc compound(s) or particle(s) thereof. Examples of such compositions and related principles are described in, e.g., US Pre-Grant Patent Application 2010/0310871 and 2013/00316582, both abandoned; see also, US Pre-Grant Patent Application Publication 2015/0376025.

The characteristics that lead to a particle being classified as mesoporous are generally known in the art. For example, their synthesis and application of mesoporous particles has been described by Baskakoti et al. “Metal-incorporated mesoporous oxides: Synthesis and applications” (J. Hazardous Materials, vol 401, 5 Jan. 2021) and by Cui et al. “Designing and Fabricating Ordered Mesoporous Metal Oxides for CO2 Catalytic Conversion: A Review and Prospect” (Materials 12(2) p.276, 16 Jan. 2019).

In aspects, zinc mesoporous particles in compositions comprise average pore diameters in the range of 2-100, 2-80, 2-50, or 2-40 (e.g., 5-50 or 5-40) nanometers. In aspects, at least some, most, generally all, essentially all, or all of the pores, in, in aspects some, most, generally all or all of such porous particles of a particle population/composition, may be filled with an amount of the associated composition/formulation.

In aspects, porous particle(s) of a particle population have a refractive index that is detectably or significantly different from that of skin. In aspects, the at least partial fill with the formulation of at least some pores of at least some of the porous particles of the porous particle population detectably or significantly modifies the refractive index of the particles such that the refractive index of the particles becomes detectably or significantly closer to that of skin.

In aspects, the at least partial fill of at least some pores with formulation of at least some of the porous particles of the porous particle population provides for a refractive index detectably or significantly closer to that of skin than the particle population having a reduced filling of pore(s) such that a composition containing such at least partially filled porous particles is detectably or significantly less whitening upon application to the skin (e.g., as compared to composition(s) where such porous particulate population(s) remain less filled or unfilled.

In certain embodiments of the present disclosure, prior to mixing ZnO with TiO₂, ZnO may be dispersed in one or more of oil, ester, triglyceride, and/or silicone fluid, preferably one or more of dimethicone, phenyl trimethicone, caprylic/capric triglyceride, or an ester selected from the group of butyloctyl salycilate and octyldodecyl neopentanoate.

Additionally, in other embodiments of the disclosure, prior to mixing ZnO with TiO₂, ZnO may also dispersed in polyhydroxystearic acid (PHSA), a polymer of hydroxystearic acid, which is commercially available under the tradename Dispersun DSP OL 300 from Innospec Performance Chemicals (Salisbury, NC). Without wishing to be bound to a particular theory, according to technical literature from Innospec, polyhydroxystearic acid may increase UV absorption of sunscreens containing pigments, such as but not limited to ZnO and TiO2, by allowing higher concentrations of pigment to be used. Additionally, the inclusion of polyhydroxy stearic acid (polyhydroxystearic acid) is described by Innospec as improving optical transparency and reducing whitening (when a finished formulation is applied to human skin). In some aspects, polyhydroxy stearic acid may function as an SPF booster in a composition.

In aspects of the disclosure, polyhydroxystearic acid may be present in a composition at a concentration of about 0.25 wt. % to about 2 wt. %, in certain aspects, at least about 0.5 wt. %.

Different molecular weights of polyhydroxystearic acid may be used in broad-spectrum compositions of the present disclosure.

Optionally, one or more additional metal oxides selected from the group of iron oxides, zirconium oxide, bismuth oxychloride and cerium oxide may be incorporated in broad-spectrum mineral sunscreen compositions of the present disclosure.

In certain embodiments, CeO₂ may be present in a broad-spectrum photoprotective composition of the present disclosure at a concentration of from about 0.1 wt. % to about 2 wt. %, in further aspects at about 0.25 wt. %.

In aspects of composition of the disclosure, cerium oxide is ARG-SPHERE NIR-1/15BA000—a combination of poly(methyl methacrylate), also known in the art as PMMA, cerium oxide (CeO₂), and aluminum oxide from Argan Co. Other available grades of Cerium oxide from Argan Co which may provide the same functionality include: ARG-Sphere NIR BP1000, ARG-Sphere NIR BP1000 AS, ARG-Sphere NIR BT1000, ARG-Sphere NIR TiO2, and ARG-Sphere NIR ZnO.

PMMA may in aspects be a spherical ultra-fine texturizing powder, available in various sizes, used in powders, among other things to, e.g., increase smoothness, fluidity and lubricity, or any combination thereof. It is understood in the art that PMMA is a polymer of methyl methacrylate, and has an empirical formula C₅H₈O₂)x.

Iron oxide(s) may be present in broad-spectrum photoprotective compositions of the present disclosure. In aspects, iron oxide(s) may be present in compositions at a concentration that is effective to achieve one or more detectable or significant effects associated with this ingredient (e.g., providing a visible tinting effect to the composition). In aspects, iron oxide(s) may be present at a concentration of up to about 7 wt. %, e.g., from about 1 wt. % to about 5 wt. %, or from about 1.5 wt. % to about 4 wt. %. In aspects, compositions comprising iron oxide(s) may further comprise pigmentary-grade titanium dioxide

[symbol]

In tinted formulations (containing one or more iron oxides selected from the group of black iron oxide, red iron oxide, and yellow iron oxide), amorphous spherical silica may, in aspects, be present at a concentration of at least 2 wt. %.

In aspects, compositions can comprise zinc oxide particles and titanium dioxide particles in an amount representing between about 10 wt. % to about 34% of the composition.

In non-tinted formulations (e.g., those not containing iron oxides), amorphous spherical silica may, in certain embodiments, be present in a composition at a concentration of at least 2.5 wt. %.

### Siliceous Compounds

Broad-spectrum photoprotective compositions of the present disclosure may contain one or more siliceous compounds that impart one or more detectable properties to the composition, such as by detectably or significantly changing the light scattering properties of the composition. In aspects, the compositions of this disclosure comprise at least two distinct siliceous compounds, which in aspects include an amorphous silica, in certain embodiments amorphous spherical silica, and material derived from one or more species of diatomaceous algae, such as compositions comprising an effective amount, of frustules derived from diatomaceous algae. In aspects, such media/frustules may possess one or more light reflecting, light refracting, light absorbing, light scattering, or otherwise light altering properties, in each case the light being UV, blue, other non-blue visible, or near-infrared wavelength, each siliceous compound described in detail below.

A siliceous ingredient of compositions can be an amorphous silica, also known in the art as amorphous silicon oxide hydrate. In aspects, the amorphous silica is a spherical powder, a porous powder, a powder having a mean particle size ranging from about 6 microns to about 10 microns, or any combination thereof. In one embodiment of the disclosure, an amorphous spherical silica is Silisphere LS-8H available from Argan Co. Other suitable amorphous spherical silica may include Silisphere 4M, Silisphere 6M, Silisphere 10M or any suitable combination thereof. Amorphous silica, in aspects amorphous spherical silica, may be present in broad-spectrum photoprotective compositions of the present disclosure at a concentration of from about 0.5 wt. % to about 5 wt %, in aspects about 3 wt. % to about 5wt. %, and in other aspects about 3 wt. % to about 4.5 wt. %.

A second siliceous composition/ingredient/component of a composition is a material from diatomaceous algae-unicellular, photosynthetic microorganisms. In aspects, the diatomaceous algae material is light reflecting, light refracting, light absorbing, light blocking, light scattering, or otherwise light altering (in all cases applied to any or all wavelengths of light from UV to near-infrared). In aspects, the material comprises, mostly comprises, or generally consists of frustules which may be derived from one or more diatom species.

In the art it is known that diatomaceous algae may have a nano-patterned cell encasement made of amorphous biosilica, also known in the art as “frustule,” that creates a highly efficient light trapping mechanism. See J. Romann et al. “Wavelength and orientation dependent capture of light by diatom frustule nanostructures” (Romann, J. et al. Wavelength and orientation dependent capture of light by diatom frustule nanostructures. Sci. Rep. 5, 17403; doi: 10.1038/srep17403 (2015)). Also see X. Chen et al., “Numerical and experimental investigation of light trapping effect of nanostructured diatom frustules” (Chen, X. et al. Numerical and experimental investigation of light trapping effect of nanostructured diatom frustules. Sci. Rep. 5, 11977; doi: 10.1038/srep11977 (2015)). See also J. Mishler et al, “Biomimetic Photonic Crystals based on Diatom Algae Frustules” presented at the March 2015 meeting of the American Phytopathological Society, Abstract #A4.004. In aspects, frustules or frustrule-containing material may be present in compositions of the disclosure from about 0.1 wt. % to about 2 wt. %

[symbol]

An example of a suitable frustule source is Plankton Glass Flower® (INCI Name: “Plankton Extract”). Frustules derived from one or more diatomaceous algal species, are commercially available from Odycea SAS (Lannion, France) and distributed in the United States by Argan Co. Technical data sheets and brochures describe Plankton Glass Flower as “algal photonic and porous silica crystals” or, alternatively, “planktonic material” sourced from the lakes in the volcanic region of Auvergne, France—namely, “siliceous fragments of freshwater algae species[,] mainly Fragilaria zeilleri, Gomphonema angustatum, Navicula radiosa, and Cyclotella andancensis”—that may in aspects contain greater than about 75 wt. % silica. Without wishing to be bound by a particular theory, it is hypothesized that Plankton Glass Flower can provide “UV-visible [light] attenuation due to both reflection and scattering” and also may serve as an “oil absorber” and “pollutant scavenger which entraps . . . impurities [at the surface of the skin]” when included in embodiments of the disclosure. In aspects, frustules may also be known as siliceous fragments to one of ordinary skill in the art. Again, not wishing to be bound by a particular theory, the supplier of algal photonic and porous silica crystals, frustules, for use in the personal care industries may create 3-dimensional structures that entraps particulate pollutants (e.g., below 2.5 microns) or attenuate/block visible blue light, or any combination thereof.

In aspect of the disclosure, frustules may be derived from one, two, three, or more diatomaceous algal species, which may have photonic and porous silica crystals and a silica content of at least about 75 wt. % is/are present in broad-spectrum photoprotective compositions of the present disclosure at a concentration from about 0.1 wt. % to about 2 wt. %, and in certain embodiments from about 0.1 wt. % to about 1 wt %.

In certain aspects, frustules from one or more diatomaceous algal species having photonic and porous silica crystals and a silica content of at least about 75 wt. % are selected from the group consisting of Fragilaria zeilleri, Gomphonema angustatum, Navicula radiosa, and Cyclotella andancensis.

In certain embodiments of the disclosure, the ratio of (i) frustules from one or more diatomaceous algal species having photonic and porous silica crystals and a silica content of at least about 75 wt. % to (ii) amorphous silica, in aspects amorphous spherical porous silica, may be from about 1:2.5 to about 1:50, about 1:10 to about 1:50, about 1:20 to about 1:50, about 1:30 to about 1:50, about, 1:2.5 to about 1:40, about 1:2.5 to about 1:30, about 1:2.5 to about 1:20, about 1:2.5 to about 1:10, or about 1:25 to about 1:45. In any aspect described herein with respect to “frustules” or “species”, such disclosure provides implicit support for a corresponding aspect comprising material/ingredient comprising such frustules or other effective material(s)/ingredient(s) derived from one or more diatomaceous algae. In aspects, compositions can comprise two siliceous compounds in an amount representing between about 0.6 wt. % to about 7 wt. % of the composition.

Total silica content in compositions of the present disclosure-e.g., silica found in (i) frustules derived from one or more diatomaceous algal species having photonic and porous silica crystals and a silica content of at least about 75 wt. % and (ii) amorphous silica, e.g., amorphous, spherical silica-may in aspects be from about 0.6 wt. % to about 7 wt. %, in certain aspects from about 2 wt. % to about 4 wt. % (e.g., 1-6%, 1-5%, 1-5.5%, 1.5-6%, 1.5-5.5%, 1.5-4.5%, 1-4%, 2-5%, 2-7%, 1.3-5%, etc.).

In some aspects, compositions of the disclosure may be tinted with one or more iron oxides, the ratio of (i) frustules, which may be derived from one or more diatomaceous algal species having photonic and, in aspects comprise porous silica crystals and a silica content of at least about 75 wt. % to (ii) amorphous silica, in aspects amorphous spherical porous silica, may be about 1:2.5 to about 1:40, about 1:10 to about 1:40, about 1:20 to about 1:40, about 1:30 to about 1:40, about 1:2.5 to about 1:30, about 1.25 to about 1:20, or about 1.25 to about 1:10.

In other embodiments of the present disclosure that are compositions that are not tinted (e.g., do not contain one or more iron oxides), the ratio of (i) frustules which may be derived from one or more diatomaceous algal species (e.g., materials having photonic and porous silica crystals and a silica content of at least about 75 wt. %) to (ii) amorphous silica compound(s) (or a component thereof), in aspects amorphous spherical porous silica, may be present in a ratio/relationship of about 1:5 to about 1:50, about 1:10 to about 1:50, about 1:20 to about 1:50, about 1:30 to about 1:50, about 1:40 to about 1:50, about 1:5 to about 1:40, about 1:5 to about 1:30, about 1:5 to about 1:20, or about 1:5 to about 1:10.

Any aspect described herein using the term “frustule” will be understood as providing implicit disclosure of corresponding aspects that are characterized as comprising a corresponding amount of a composition/ingredient/component including an effective amount, concentration, or number of frustules or corresponding diatom algae structures, mostly comprising frustules, generally consisting of frustules, substantially consisting of frustules, consisting essentially of frustules, or consisting of frustules.

### Film-Forming Polymers and Silicone Compounds

In aspects, compositions of the disclosure include a film-forming component, comprising one or more film-forming compounds. Film-forming ingredients are generally understood in the art to be ingredients that may produce a continuous film on skin.

One type of composition that can function as a film-forming agent/ingredient is a film-forming siloxane. Siloxanes, also known in the art as organo-substituted polysiloxanes, are linear or cyclic polymers of monomeric silicon/oxygen monomers, in which a polymeric backbone is made up of alternating silicon and oxygen atoms. The silicon atoms may carry a wide variety of substituents, which can be the same or different.

Compositions of the present disclosure in aspects have a water resistance of 80 minutes and may be comprised of one, two, or more, film-forming polysiloxane polymers.

In certain embodiments, two film-forming polymers may comprise, mostly comprise, generally consist of, substantially consist of, consist essentially of, or consist only of (i) Bis-Vinyl dimethicone/dimethicone copolymer, which can be commercially available in combination with dimethicone, cetyl dimethicone, or phenyl dimethicone and (ii) Dimethicone (and) acrylates/dimethicone copolymer. It is understood in the art that bis-vinyl dimethicone/dimethicone copolymer is a copolymer of dimethicone end-blocked with vinyl dimethicone. It is also understood in the art that dimethicone (C₂H₆OSi)xC₄H₁₂Si is a mixture of fully methylated linear siloxane polymers end blocked with trimethylsiloxy units. It is understood in the art that vinyl dimethicone is a derivative of dimethicone in which some of the methyl groups are replaced with vinyl groups. The vinyl groups can occur at the ends of the siloxane chain may be pendant to the siloxane chain. It is also understood in the art that bis-vinyl dimethicone is a derivative of dimethicone in which one methyl group at each end of the siloxane chain is replaced with a vinyl group. It is understood in the art that cetyl dimethicone is a dimethyl siloxane. It is also understood in the art that acrylates/dimethicone copolymer is a copolymer of dimethicone and one or more monomers of acrylic acid, methacrylic acid or one of their simple esters.

In aspects of the disclosure, in addition to at least two dimethicone copolymer film formers (discussed immediately above), the broad-spectrum photoprotective compositions of the disclosure may be comprised of at least two, in some embodiments three silicone compounds selected from dimethicone, simethicone, and phenyl trimethicone.

In certain embodiments, a first silicone compound is a silicone elastomer (in aspects dimethicone/vinyl dimethicone crosspolymer), and a second silicone compound may be a silicone fluid (in aspects selected from the group of dimethicone, simethicone, and phenyl trimethicone).

It is understood in the art that dimethicone is a mixture of fully methylated linear siloxane polymers end-blocked with trimethylsiloxy units.

In certain embodiments, dimethicone may be present in combination with trimethylsiloxysilicate/dimethicone crosspolymer, and in further combination with one or more esters of stearic acid (e.g., a polyethylene glycol ester of stearic acid or a monoester of glycerin and stearic acid).

The following are examples of such types and other types of ingredients that can be suitably included in compositions.

Mixtures of dimethicone, trimethylsiloxysilicate/dimethicone crosspolymer, and two esters of stearic acid are commercially available from Dow Corning (Midland, Michigan) under the tradenames Xiameter™ AFE-0020 and Xiameter™ AFE-3101. Simethicone is a mixture of dimethicone with an average chain length of 200 to 350 dimethylsiloxane units and silica, e.g., Xiameter™ AFE-1510, and can, in aspects, act as an antifoaming agent. Xiameter™ AFE-1510, as currently available in the market, is described as being 10% active food-grade silicone emulsion (silicones and siloxanes, dimethyl) with the balance being about 90% water. This type of ingredient exemplifies how compositions of the invention can contain 1, 2, 3, 4, or 5 ingredients that contain water, but when combined with other ingredients of the composition, still result in a composition that is characterizable as a low water content composition, e.g., a composition in which the total water content of the composition remaining under ˜5%, under ˜4.5%, under 4%, under ˜3.75%, under 3.5%, under 3.3%, under ˜3.1%, under 2.8%, or under ˜2.6% water.

It is understood in the art that phenyl trimethicone is a siloxane polymer. Phenyl trimethicone in aspects may be used in combination with bis-vinyl dimethicone/dimethicone copolymer, and is commercially available from Jeen International (Fairfield, NJ) under the tradename JEESILC® PTMF-405.

In aspects compositions of the invention can include a hectorite compound/composition, such as, e.g., disteardimonium hectorite. It is understood in the art that disteardimonium hectorite is the reaction product of (a) hectorite, known in the art to be one of the montmorillonite minerals that are the principal constituents of bentonite clay and (b) distearyldimonium chloride, a quaternary ammonium salt. Disteardimonium hectorite is commercially available under the tradename Bentone® 38V from Elementis Specialties (East Windsor, NJ).

Without wishing to be bound by a theory, the Applicant believes that inclusion of the above-described film-forming polymers, silicone compounds, or clay compositions, alone or in combination, may, in aspects, provide aesthetic and performance advances not achieved in the prior art e.g., ease of application (spreadability), non-tacky and non-oily after-feel, without leaving a white residue on Fitzpatrick Skin Types I-IV, or any combination thereof.

### Emollient Component/Emollients

In aspects, at least about 25%, at least about 33%, or at least about 40% of the ingredients of a composition can be characterized as emollients (or as being made up of an emollient component) on a weight percent basis (e.g., 20-60%, 20-55%, 20-50%, 25-55%, 25-50%, 30-55%, 30-50%, or 30-45% of the composition can be composed of emollient(s)). Examples of emollients that can be included in a composition include compositions/ingredients that mostly, generally only, substantially only, essentially only, or entirely are composed of Caprylic/Capric Triglyceride(s), Dimethicone, Bis-Vinyl Dimethicone/Dimethicone Copolymer, Phenyl Trimethicone, Bis-Vinyl Dimethicone/Dimethicone Copolymer, octyldodecyl neopentanoate, or butyloctyl salicylate. Readers will understand that use of capitalization of terms or other form elements of terms (e.g., whether such terms are presented as one word or two) can vary herein or vary herein from usage in the art without changing the meaning of such terms.

### Waxes

Broad-spectrum photoprotective compositions of the present disclosure may, in aspects, include any suitable number of waxes (e.g., one, two, three, or more waxes), in any suitable/effective amount(s), which, in aspects, can be selected from the group of waxes that are characterizable as “natural,” “mineral,” “synthetic,” or “petroleum” in origin. Suitable waxes in certain embodiments of the composition mostly, generally entirely, substantially entirely, or entirely do not recrystallize or “bloom” when the sunscreen composition undergoes accelerated stability testing (e.g., at 40° C. or 50° C. for about or over three or more months) or after three (3) or more freeze-thaw cycles.

It is generally understood that natural waxes may be botanical or animal in origin. In aspects, natural waxes may be botanical waxes that, in certain embodiments, may include candelilla wax, castor wax, cotton wax, soy wax, jojoba wax, olive wax, carnauba wax, sugar cane wax, rice bran wax, bayberry wax, sunflower wax, rose petal wax, and Japan wax, or any combination thereof.

In aspects, natural waxes can be comprised of a fixed oil expressed or extracted from the kernel of a number of plant varieties and/or unsaponifiables within the kernel oil. Fixed oils are readily understood by one of ordinary skill in the art, e.g., as described by Apte “Fixed-Oil Excipient Monographs: Development of USP Fixed-Oil Reference Standards” (Pharm. Technology 37(4) 2 Apr. 2013). In certain aspects of the disclosure, the kernel oil and unsaponifiables within the kernel oil, may be hydrogenated.

In certain embodiments of the disclosure, a natural wax in a composition may be Albiwax (from Argan Co.), which is a mixture of Prunus armeniaca (apricot) kernel oil, hydrogenated apricot kernel oil, unsaponifiables of apricot kernel oil, and hydrogenated unsaponifiables of apricot kernel oil. Natural waxes of certain embodiments of the disclosure may be of animal origin and may include, but are not limited to, beeswax.

In aspects, mineral waxes that can be present in a composition of the invention include montan wax, ozokerite, and ceresin. Of the mineral waxes, ozokerite—a hard, high melting point wax blend consisting of branched and linear hydrocarbons—may be contained in certain embodiments of the disclosure. It is recognized in the art of formulating personal care products that ozokerite may vary in a number of important respects, which can impact aesthetics/structure and quality/duration of a film deposited on the skin, among other respects. These variations may include melting point, congeal point, degree of penetration, or any combination thereof. Ozokerite 2095, available from Koster Keunen (Watertown, CT), may be contained in certain embodiments of the disclosure and is known in the art to have a maximum degree of penetration of 5 dmm as measured by ASTM Standard D1321-16a (standard test method for needle penetration of petroleum or mineral waxes, e.g.).

Synthetic waxes that can be included in compositions of the invention in certain embodiments of the disclosure may include polyethylene waxes, silicone waxes, fluoro waxes, Fischer-Tropsch waxes, polypropylene waxes, esters of poly(ethylene glycol), and pegylated sorbitans, alone or in combination with, for example, monoalkyl ethers of poly(ethylene glycol) (e.g., ceteareth-20).

It is known in the art that polyethylene is a polymer of ethylene monomers that conforms generally to the formula: (C₂H₄)x. Also known in the art, polyethylenes may vary in melting-point and hardness, and other attributes depending on hydrocarbon distribution (e.g., linear versus branched). Such materials can be included in compositions of the invention. In one embodiment, polyethylene used in broad-spectrum photoprotective compositions of the present disclosure may be a linear polyethylene with a melting point of from about 80° C. to about 90° C. In certain aspects, a non-limiting example of a linear polyethylene meeting this criterion is JEENATE® 5H from JEEN International.

It is known in the art that synthetic waxes may also include so-called “functionalized waxes,” non-limiting examples of which are pegylated animal waxes (e.g., PEG-8 beeswax). Such ingredients can be included in compositions in aspects.

Petroleum-based waxes (also known in the art as mineral waxes) suitable for use in the compositions of the present disclosure may include paraffin wax and microcrystalline wax.

In certain embodiments, the compositions of the present disclosure may include at least one natural wax, in further aspects of botanical origin, and at least one mineral wax.

In aspects, compositions of the present disclosure may include at least one “soft” wax (preferably natural) and at least one “hard” wax, which can be a mineral wax or synthetic wax or both. By “soft,” it means the wax has a melting point of from about 60° C. to about 70° C. By “hard,” it means the wax has a melting point of from about 80° C. to about 90° C.

In certain embodiments, waxes of a composition may include tribehenin and polyethylene. It is understood in the art that tribehenin is the triester of glycerin and behenic acid. Commercially available under the tradename Syncrowax™ HRC from Croda Inc., tribehenin is a non-crystalline, “soft” wax, having a melting point of from about 60° C. to about 70° C., and may be used for among other things to modify crystallinity and rigidity of other waxes, provide a “creaminess” and spreadability to the sunscreen composition, or any combination thereof.

Broad-spectrum photoprotective compositions of the present disclosure may be comprised of at least two waxes (e.g., 2-4 distinct waxes, 2-5 distinct waxes, 3-6 distinct waxes, or 3-5 distinct waxes) in a combined concentration of from about 7 wt. % to about 15 wt. % (e.g., 8-12, 7.5-12.5, 7.5-15, 9-15, 9-12, 10-15, 7-12.5, 7-10, or 9-14%), and in certain aspects from about 10 wt. % to about 13 wt. % (e.g., 10.5-12.5% or 11-12%).

In aspects, at least one wax of the wax component of a composition is a polycyclic hydrocarbon or carbohydrate wax, comprising at least 3, 4, or 5 cyclopentyl/cyclopentane or cyclohexyl/cyclohexane rings and, in aspects, comprising at least one oxygen. In aspects, the polycyclic hydrocarbon or carbohydrate wax has a molecular weight of about 250-1000, e.g., about 300-600, e.g., about 400-500. In aspects, most of the mass of the polycyclic hydrocarbon or carbohydrate wax is contained in the polycyclic ring structures of the wax. An example of a polycyclic carbohydrate wax exhibiting a combination of the above-described features is ozokerite. In aspects, a polycyclic wax makes up about

[symbol]

In aspects, most, generally all, substantially all, or all of the waxes of a wax component are amorphous waxes.

In some aspects, some, of the wax component of a composition (e.g., about 1-5% of the composition, about 1.5-6% of the composition, about 1.75-6% of the composition, or about 1.75%-5.5% of the composition) comprise a linear or branched hydrocarbon/carbohydrate structure. In aspects, such linear/branched waxes account for about 15-65%, 25-65%, 20-70%, 22.5-57.5%, 27.5-52.5%, 35-55%, 40-60%, 40-55%, or 30-55% of the wax component of the composition. Examples of such waxes include polyethylene waxes and Tribehenin wax (2,3-di(docosanoyloxy)propyl docosanoate). In aspects, at least one of the wax compounds of the wax component (e.g., at least about 0.5-3% of the composition, such as 0.75-2.75% or 1-2.5% of the composition) comprises a glycerol/glycerin/glycerol-like structure (e.g., like Tribehenin). In aspects, some, most, or all of the linear/branched structure wax compounds of the composition have a molecular weight of at least 750 (e.g., about 800-8000, 900-6300, or ˜950-5600, etc.).

In aspects, the ratio of mineral wax, e.g., ozokerite, to the total amount of wax in the disclosed compositions can be about 1:1.5 to about 1:4 (e.g., about 1:2 to about 1:4 or about 1:2 to about 1:3), in certain aspects can be about 1:2.

In aspects, the ratio of polycyclic wax compound(s) (component) to linear/branched wax compound(s) (component) in the composition is about 0.5:1 to about 2:1, such as about 0.75:1 to about 1.5:1, e.g., about 0.8:1 to about 1.2:1, e.g., about 0.9:1 to about 1:1.1.

### Water Content

In some aspects, the compositions of the invention contain very little water or no detectable amount of water.

In aspects, compositions of the present disclosure may include a detectable amount of water. In aspects, some, most, generally all, substantially all, or all the water in the composition is from water contained in components/ingredients that are part of the composition (that are used to form the compositions).

In certain embodiments, water may be present in an amount of about 0.1% to about 5%, about 0.5% to about 5%, about 1% to about 5%, about 2% to about 5%, or about 3% to about 5% of the composition by weight. In certain embodiments, water is about 0.1% to about 4%, about 0.1% to about 3%, about 0.1% to about 2%, about 0.1% to about 1%, or about 0.1% to about 0.5% of the composition by weight.

In certain embodiments, the composition is about 95% to about 99.9%, about 96% to about 99.9%, about 97% to about 99.9%, about 98% to about 99.9%, about 99% to about 99.9%, or about 99.5% to about 99.9% water-free by composition weight. In certain embodiments, the composition is about 95% to about 99.7%, about 95% to about 99%, about 95% to about 98%, or about 95% to about 97% water-free by weight of the composition.

In certain embodiments, a composition of this disclosure may be considered “substantially anhydrous,” which, in aspects, means that the composition is no more than 5% by weight, no more than about 3%, 2.5%, or 2% by weight, e.g., no more than about 1.5% or about 1% by weight, or even no more than about 0.5% by weight, such as no more than 0.4% by weight, or 0.35%, 0.3%, or 0.2% by weight, and, in cases about 0.1% by weight based on weight composition. It should be noted that this amount of water in aspects refers to free water and does not include, e.g., bound water, such as the water of crystallization of the salts. In aspects, this amount of water refers to the amounts of water in ingredients used to generate the composition and does not include water from other sources, such as, e.g. traces of water absorbed by the raw materials, amounts of water absorbed during production of the composition, ingress of water from the atmosphere during storage of the composition, or the like.

### SPF Booster(s)

In aspects, compositions disclosed herein comprise one or more ingredients that are classifiable as SPF boosters. In aspects, the SPF boosters are compound(s)/ingredient(s) that detectably or significantly increase SPF but don't alone contribute significantly to the SPF of the compositions.

Broad-spectrum photoprotective compositions of the present disclosure in aspects may contain one, two, and even three or more, typically long chain, branched compounds, which may boost SPF, such as compounds selected from the group of butyloctyl salicylate (described below), octyldodecyl neopentanoate (described below), and polyhydroxystearic acid (described above).

Butyloctyl salicylate (CAS No. 190085-41-7), commercially available as HallBrite® BHB from the Hallstar Company (Chicago, Illinois) and SunSolv® from Innospec Performance Chemicals (Salisbury, North Carolina) is a synthetically produced ester of salicylic acid and a branched C₁₂ alcohol, 2-butyloctanol. Butyloctyl salicylate, and its uses in topical formulations, are described in the following U.S. Pat. Nos. 5,783,173; 5,788,954; 5,849,273; and 6,350,894.

Octyldodecyl neopentanoate is the ester of octyldodecanol (q.v.) and neopentanoic acid. It is sold under the tradename Elefac 1-205 by Alzo International Inc. (Sayreville, NJ).

In aspects, compositions can comprise at least one ingredient which significantly increases in vivo SPF, but doesn't alone contribute significantly to the SPF, aka SPF booster, in an amount representing between about 0.25 wt. % to about 42 wt. % of the composition.

Surprisingly and unexpectedly, broad-spectrum photoprotective compositions of the present disclosure may contain one, two, or three of the above SPF Boosters to provide an SPF of at least 30, and, in certain embodiments, an SPF of at least 50, without visible whitening on Skin Types I-IV under the Fitzpatrick Skin Type system of skin classification, e.g., as described in Appendix A.

### Powders and Micas

Broad-spectrum photoprotective compositions of the present disclosure preferably reduce the visible signs of aging through a synergistic complex of optical blurring powders comprised of: (a) two siliceous compounds-namely, an amorphous spherical silica and frustules derived from a mixture of diatomaceous algal species, each having photonic and porous silica crystals (Plankton Glass Flower®), as described above, in further combination with (b) at least one vinyl dimethicone crosspolymer, selected from the group of dimethicone/vinyl dimethicone crosspolymer and vinyl dimethicone/methicone silsesquioxane crosspolymer.

Vinyl dimethicone/methicone silsesquioxane crosspolymer is commercially available under the tradename KSP-101 from Shin-Etsu Silicones of America (Akron, Ohio). Such agents, like any compounds/ingredients described herein, uncontradicted, can be present in compositions in suitable/effective amounts. Dimethicone/vinyl dimethicone crosspolymer may be combined with at least one polyethylene glycol ether of lauryl alcohol. Such materials can be present in compositions of the invention.

In certain embodiments, dimethicone/vinyl dimethicone crosspolymer may combined with laureth 3 and laureth 25, which is commercially available from Access Ingredients (South Pasadena, California) under the tradename AccessSIL EMUL-6081.

In certain embodiments, broad-spectrum photoprotective compositions of the present disclosure may include one or several mica(s), and/or mica-like compound(s), that impart shimmer, glow, reduces the appearance of skin imperfections, or any combination thereof.

In one aspect of the disclosure, a mica-like compound is included in the composition and in aspects the mica-like compound comprises or is Synthetic Fluorphlogopite, a synthetic mineral that conforms generally to the formula:

Mg₃K[AlF₂O(SiO₃)₃].

In certain embodiments, mica (or a mica-like compound) may be present in combination with iron oxide(s), titanium dioxide, or both.

### Additional Cosmetic Skin Benefit Ingredients

Broad-spectrum photoprotective compositions of the present disclosure may include one or several oil-soluble ingredients that (a) reduce visible redness (i.e., erythema) or inflammation (known in the art as anti-inflammatory) or act as an antioxidant (i.e., reduce oxidative damage; also known in the art as free radical quenchers), (b) reduce the appearance of the signs of skin aging, which can include fine lines, wrinkles, uneven pigmentation (dyschromia), loss of elasticity or firmness, increased dryness, reduced skin moisture, loss of softness/suppleness (collectively “anti-aging ingredients”), (c) oil-absorbent powders, or (d) any combination thereof. Non-limiting examples of antioxidants and anti-aging ingredients may include vitamins and derivatives thereof, in certain aspects, tocopherol (vitamin E) esters; proteins or compounds that stimulate elastin gene expression and/or downregulate collagenase gene expression (e.g., matrix metalloproteinases, “MMPs”); as well as ingredients known in the art as humectants, moisturizers, skin-conditioning agents, skin soothing and/or healing agents.

Non-limiting examples of ingredients that may quench reactive oxygen species (ROS) stimulate elastin gene expression and/or downregulate collagenase (MMP) gene expression include bioflavonoids; amino acids; peptides, in certain embodiments comprised of about two to about ten amino acids, in further embodiments lipidated peptides; stem cells, including stem cell lysates; glycyrrhizinates; and ceramides (or ceramide-like compounds), or any combination thereof.

In aspects, antioxidants/radical scavengers/moisturizing agents may be topically delivered in the broad-spectrum photoprotective compositions of the present disclosure may include: bisabolol; algal extracts of Himanthalia elongata (Sea Spaghetti); Zingiber officinale Root Extract, cetylhydroxyproline palmitamide, or any combination thereof.

Himanthalia elongata Extract is commercially available under the tradename Marine Bamboo TG from Argan Co., at a concentration of 5-25% (on an active basis) in a carrier of caprylic/capric triglyceride.

Cetylhydroxyproline palmitamide is commercially available from Symrise (Teterboro, NJ) in mixture with other skin benefit ingredients under the tradenames SymRepair® and SymCare®. Both of these mixtures contain bisabolol and Cetylhydroxyproline palmitamide and Brassica campestris (Rapeseed) sterols.

Non-limiting examples of oil-absorbent powders include Nylon-12 (a polyamide derived from 12-aminododecanoic acid) and polymethylsilsesquioxane (a polymer formed by the hydrolysis and condensation of methyltrimethoxysilane).

In certain embodiments, Nylon-12 may be combined with polymethyl methacrylate or polymethylsilsesquioxane and included in compositions.

Certain embodiments of the present disclosure include ingredients that may act as pollutant scavengers (i.e., entrap or reduce the negative effects of environmental particulate matter on skin). “Negative effects on the skin” means, e.g., DOS collagen degradation, overproduction of melanin, inflammation, or any combination thereof. Non-limiting examples of preferred pollutant scavengers that can be included in effective amounts in compositions may include Plankton Glass®, Himanthalia elongata extract, or both.

Compositions of the present disclosure may in aspects contain one or several ingredient(s) that in any combination absorb, attenuate, reduce negative effects on the skin caused by blue light, near-infrared, and/or infrared radiation.

In aspects of the disclosure, an ingredient that scatters infrared radiation is a combination of PMMA, cerium oxide, aluminum oxide, available from Argan under the tradename ARG-NIR.

In certain aspects, compositions of the present disclosure do not contain any of the following: paraben, formaldehyde, chlorphenesin, and phenoxyethanol. In certain aspects, preservative systems of compositions of the disclosure may include hydroxyacetophenone (in aspects at 0.5%), a mixture of 1,2 Hexanediol (and) caprylyl glycol (in aspects at a combined concentration of 0.5%), or a mixture of bisabolol (and) Zingiber officinale (Ginger) Extract (in aspects at a combined concentration of 0.1%).

### Ingredients That may be Characterized Based on Proportional Relationship(s) to Other Ingredients (Weight Percent or Other Ratios)

In aspects, compositions can comprise, e.g., at least one ingredient that significantly increases in vivo SPF, but doesn't alone contribute significantly to the SPF of the composition (i.e., boosters), one or more, e.g., at least two siliceous compounds, and zinc oxide particles and titanium dioxide particles, wherein these ingredients are present in certain defined amounts, e.g., as provided in this section or elsewhere, or that maintain a desired ratio with respect to one another.

For example, a composition can comprise such ingredients in amounts according to the following ranges.

| Compound/ingredient(s) | Low amount | High amount |
|---|---|---|
| SPF booster | 0.25% | 42% |
| Two siliceous compounds | 0.6% | 7% |
| Zinc oxide + titanium dioxide | 10% | 34% |

In certain aspects, compositions can comprise e.g., at least one ingredient that significantly increases in vivo SPF, but doesn't alone contribute significantly to the SPF of the composition (i.e., an SPF booster), at least two siliceous compounds, and zinc oxide particles and titanium dioxide particles in relative amounts such as relative amounts defined by one or more ratios created by values provided in the table above.

As a non-limiting example, selecting a value based on the guidance in the table provided above, at least one SPF booster may be present in an amount of about 0.25%, two or more siliceous compounds may be present in an amount of about 3%, and zinc oxide and titanium dioxide may be present in an amount of about 34%. This would, e.g., correspond to a formulation wherein the ratio of SPF booster to two or more siliceous compounds to zinc oxide+titanium dioxide is 0.25:3:34. Compositions can comprise other suitable amounts of these materials in such or similar ratios.

As an alternative non-limiting example, selecting high values for all components, at least one SPF booster may be present in an amount of 42%, two siliceous compounds in an amount of 7%, and zinc oxide and titanium dioxide in an amount of 34%. This would correspond to a ratio of SPF booster to two or more siliceous compounds to zinc oxide+titanium dioxide of 42:7:34. Compositions can comprise other suitable amounts of these materials in such or similar ratios.

The following table provides a summary of non-limiting exemplary relationships defined by ratios and ranges of amount/weight percentage/concentration between the 3 components/ingredients provided wherein the order of the components of the ratio is SPF booster:siliceous compounds:ZnO+TiO₂:

| | | | | | |
|---|---|---|---|---|---|
| 0.25:0.6:10 to | 0.25:0.6:10 to | 0.25:0.6:10 to | 0.25:0.6:10 to | 0.25:0.6:10 to | 0.25:0.6:10 |
| 0.25:0.6:34 | 0.25:7:10 | 0.25:7:34 | 42:0.6:10 | 42:7:10 | to 42:7:34 |
| X | 0.25:0.6:34 to | 0.25:0.6:34 to | 0.25:0.6:34 to | 0.25:0.6:34 to | 0.25:0.6:34 |
| | 0.25:7:10 | 0.25:7:34 | 42:0.6:10 | 42:7:10 | to 42:7:34 |
| X | X | 0.25:7:10 to | 0.25:7:10 to | 0.25:7:10 to | 0.25:7:10 to |
| | | 0.25:7:34 | 42:0.6:10 | 42:7:10 | 42:7:34 |
| X | X | X | 0.25:7:34 to | 0.25:7:34 to | 0.25:7:34 to |
| | | | 42:0.6:10 | 42:7:10 | 42:7:34 |
| X | X | X | X | 42:0.6:10 to | 42:0.6:10 to |
| | | | | 42:7:10 | 42:7:34 |
| X | X | X | X | X | 42:7:10 to |
| | | | | | 42:7:34 |

These examples are provided for illustration purposes only. Other suitable ratios may be derived using any value between and including the endpoints for each component/ingredient(s) provided in this section or in other parts of this disclosure to define suitable ratios of ingredients that can be used to characterize compositions of the invention.

### EXAMPLES

The disclosure is further illustrated by reference to the exemplary formulation specification provided in the following table.

| Phase | Ingredient(s) | Range |
|---|---|---|
| A | Butyl Octyl Salycilate | 10.00-20.00 |
| A | Octyldocecyl Neopentanoate | 2.00-12.00 |
| A | Caprylic/Capric Triglyceride | 2.00-12.00 |
| A | Phenyl Trimethicone, Bis-Vinyl | 10.00-20.00 |
| | Dimethicone/Dimethicone Copolymer | |
| A | Polyhydroxystearic Acid | 0.10-2.00 |
| A | Dimethicone, Bis-Vinyl Dimethicone I | 2.00-10.00 |
| | Dimethicone Copolymer | |
| A | Zinc Oxide | 10.00 |
| B | Titanium Dioxide Pigmentary, Mica | 3.00-25.00 |
| | and Iron Oxides | |
| C | Caprylic/Capric Triglyceride, | 12.00-15.00 |
| | Titanium Dioxide, Alumina, Stearic | |
| | Acid (and) Polyhydroxystearic Acid | |
| D | Prunus Armeniaca (Apricot) Kernal | 1.00-5.00 |
| | Oil (and) Hydrogenated Vegetable | |
| | Oil (and) Tocopherol | |
| D | Ozokerite | 1.00-10.00 |
| D | Polyethylene | 1.00-5.00 |
| D | Tribehenin | 0.50-5.00 |
| E | Disteardimonium Hectorite | 0.10-3.00 |
| F | 1,2 Hexanediol and 1,2 Octanediol | 0.10-1.00 |
| F | Dimethicone (and) Acrylates/ | 2.00-10.00 |
| | Dimethicone Copolymer | |
| F | Dimethicone I Vinyl Dimethicone | 0.50-5.00 |
| | Crosspolymer (and) Laureth-3 (and) | |
| | Laureth-25 | |
| G | Silica | 1.00-7.50 |
| G | Plankton Extract | 0.10-2.00 |
| G | Vinyl Dimethicone/Methicone | 1.00-10.00 |
| | Silsesquioxane Crosspolymer | |
| G | PMMA, Cerium Oxide, Aluminium | 0.10-2.00 |
| | Oxide | |
| H | Simethicone | 0.10-2.00 |
| H | Phenethyl Alcohol and Caprylyl | 0.10-1.00 |
| | Glycol | |

The exemplified formulation can be prepared as follows: Combine Phase A ingredients except ZnO, mixing while heating to 65-70° C. Add ZnO; then mix and homogenize. Add Phase B ingredients; homogenize. Add TiO₂ (Phase C). Add Phase D ingredients while heating to a temperature sufficient to melt and disperse waxes. Add Phase E. Sequentially, add Phase F, G, and H ingredients.

This exemplary formulation can be further modified to conceal skin imperfections, including blemishes, fine lines, wrinkles, and uneven pigmentation by increasing the amount of coated Pigmentary Grade Titanium Dioxide to a level of up to about 20%. The ability of such a modified formulation is described in the art as improved skin coverage or “payoff’. As will be appreciated by the person having ordinary skill in the art, increasing the amount of titanium dioxide will increase coverage and the ability to conceal skin imperfections. For example, in concealer embodiments, pigmentary titanium dioxide can be present from about 5% to about 20%.

The synergistic combination of ZnO and TiO₂ with (a) two SPF Boosters—butyl octyl salycilate and neopentyl glycol (INCi: octododecyl neopentanoate; commercially available under the tradename ELEFAC® I-205, and (b) two Siliceous Compounds—an amorphous spherical silica and frustules derived from a mixture of diatomaceous algal species, each having photonic and porous silica crystals (Plankton Glass Flower®, “PGF”), e.g., in the ratios described above, can achieve a critical wavelength (“CW”) of at least 370 nm necessary to qualify as a “broad-spectrum” (UVA/UVB) sunscreen under applicable FDA regulations (as of the filing date of the present application).

In the examples, the combination of mica/mica-like and iron oxides can range from about 0.1 wt. % to about 15 wt. %. In aspects, iron oxides may be coated. In certain aspects, coated iron oxides may be iron oxide, CI 77491; iron oxide, CI 77499 and iron oxide, CI 77492, each surface treated/coated with triethoxycaprilylsilane or hydrogenated lecithin.

The following ingredients may be added to the Example, preferably to Phase H, in the indicated concentration ranges: bisabolol and Zingiber officinale (Ginger) Root Extract—0.1 wt. %); caprylic/capric triglyceride and Himanthalia elongata Extract (0.1 wt. %-5 wt. %); hexyldecanol, bisabolol, cetylhydroxyproline palmitamide, stearic acid, Brassica campestris (Rapeseed) Sterols (0.1 wt. %-5 wt. %); PMMA, cerium oxide, aluminum oxide (0.1 wt. %-2 wt. %); tocopheryl acetate (0.1 wt. %-1 wt. %); tocopheryl acetate (and) ubiquinone (0.25 wt. %-2 wt. %).

The disclosure provides a further example of a group of compositions according to the principles of this disclosure, wherein the following components are present in the indicated weight percentage and exhibit the indicated function(s):

| | Detectable or | Amount |
|---|---|---|
| Component | Significant Function(s) | (wt. %) |
| Caprylic/Capric Triglycerides | Anti-aging, even tone, anti- | 0.1-4% |
| and Himanthalia elongata | pollution, antioxidant, UV, | |
| Extract | blue, and infrared light | |
| | protection, targeted action | |
| | on elastin, MMP1 and | |
| | contractile forces in | |
| | fibroblasts | |
| Butyloctyl salicylate | Emollient, SPF booster, | 10-30% |
| | inorganic pigment wetter | |
| Octyldodecyl neopentanoate | Emollient, SPF booster | 3-10% |
| Dimethicone, bis-vinyl | Film-forming polymer | 2-6% |
| dimethicone/dimethicone | | |
| copolymer | | |
| Phenyl trimethicone, | Film-forming polymer | 5-20% |
| Dimethicone/bis-vinyl | | |
| dimethicone copolymer | | |
| Caprylic/Capric triglyceride | Emollient | 2-7% |
| Polyhydroxystearic acid | Dispersant of pigments, | 0.25-2% |
| | film former, SPF booster | |
| Zinc oxide | Pigment, inorganic sunscreen | 5-15% |
| Caprylic Capric Triglyceride, | Pigment, coated inorganic | 5-15% |
| Titanium Dioxide, Aluminum | sunscreen | |
| Hydroxide, Stearic Acid, | | |
| Polyhydroxystearic Acid | | |
| Titanium dioxide, | Pigment | 0-4% |
| triethoxycaprylylsilane | | |
| Iron oxide | Pigment, blue light and | 0-7% |
| | infrared light protectant | |
| Disteardimonium hectorite | Suspending agent | 0.25-1% |
| Bisabolol, Zingiber officinales | Anti-oxidant, radical | 0.01-1% |
| extract | scavenger, moisturizer, | |
| | anti-irritant | |
| Hexyldecanol, Bisabolol, | Skin conditioner | 0.5-2% |
| Cetylhydroxyproline | | |
| Palmitamide, Stearic Acid, | | |
| Brassica campestris Sterols | | |
| Dimethicone, dimethicone/ | Film-former | 0.5-5% |
| acrylate copolymer | | |
| Vitamin E acetate | Anti-oxidant, radical quencher, | 0.05-1% |
| | anti-aging | |
| 1,2 hexanediol, caprylyl | Preservative booster | 0.25-0.75% |
| glycol | | |
| Natural wax | Wax | 1-3% |
| Synthetic/Petroleum wax | Wax | 2-6% |
| Mineral wax | Wax | 4-6% |
| Dimethicone/Vinyl | Soft-focus powder | 0.25-0.75% |
| Dimethicone Copolymer, | | |
| Laureth-3, Laureth-25 | | |
| Vinyl Dimethicone/ | Soft-focus powder | 2-4% |
| Methicone Silsesquioxane | | |
| Crosspolymer | | |
| Water, Simethicone Xiameter | Anti-foam | 0.1-0.3% |
| AFE-1510 | | |
| Phenethyl alcohol, caprylyl | Anti-microbial, odor masker | 0.01-0.1% |
| glycol, feniol | | |
| Plankton glass flower ® | UV, blue and visible light | 0.1-2% |
| (Argan) (siliceous compound) | protectant, anti-pollution, | |
| | absorber, soft focus | |
| Amorphous porous spherical | Coating agent, light scatter, | 0.5-5% |
| silica (Silisphere LS 8H, | absorbent, soft focus | |
| Argan) (siliceous compound) | | |
| Cerum oxide, Aluminum | Anti-aging, UV, visible and | 0.1-2% |
| oxide, poly(methyl methacrylate) | infrared light protectant, provide | |
| (Arg Sphere NIR-1/15BA00) | smoothness, fluidity, lubricity | |
| Water | Hydration | 0.3 to 5% |
| Butyloctyl salicylate + | SPF Booster | 0.25 to 42% |
| octyldodecyl neopentanoate + | | |
| polyhydroxystearic acid | | |
| (minimum to maximum | | |
| amounts) | | |
| Zinc oxide + titanium dioxide | Sunscreen, pigment | 10 to 34% |
| Siliceous compounds | UV, blue, and visible light | 0.6 to 7% |
| | protectant, anti-pollution, | |
| | absorber, soft focus, Coating | |
| | agent, light scatter | |

Possible additional characteristics of such compositions can include one, some, most, generally all, substantially all or all of the following:

| | |
|---|---|
| Ratio of SPF booster to | 20:3:20 to |
| siliceous compounds to | 30:5:30 |
| total zinc oxide plus | |
| titanium dioxide | |
| SPF | >=30 |
| Critical wavelength | >370 nm |
| Water resistance in | Yes, >80 |
| accordance with standards | minutes. |
| set out in Section 352.76 | |
| of Title 21 of the U.S. Code | |
| of Federal Regulations | |
| whitening on Fitzpatrick | No |
| Skin TypeI-IV | |
| Emulsion | No |
| Blue light protection | Yes |
| Infrared light protection | Yes |

These examples are representative and should not be construed to limit the scope of the disclosure.

In aspects, compositions of the disclosure (a) lack 1, 2, 3, 4, 5, 6, 7 or more of the above-referenced ingredients, (b) comprise ingredients listed above but in 1, 2, 3, 4, 5, 6, 7, or more cases in amounts that differ from the amounts provided above, (c) include ingredients other than those listed in the above-provided tables, or (d) vary in 1, 2, 3, 4, 5, 6, 7, or more function(s) from those described above.

In aspects compositions of this disclosure comprise more than 0.4% water, such as 0.5% or more, 0.6% or more, 0.7% or more, or 1% or more water, but less than 5%, less than 4.5%, less than 4.2%, less than 4%, less than 3.75%, less than 3.5%, or less than 3% water (e.g., comprise 0.5-3.5% water, comprise 0.6-3.75% water, or comprise 0.7%-4.2% water, etc.).

While the illustrative embodiments of the disclosure have been described with particularity, it will be understood that various other modifications will be apparent to and can be readily made by those skilled in the art without departing from the spirit and scope of the disclosure.

Accordingly, it is not intended that the scope of the claims appended hereto be limited to the examples and descriptions set forth hereinabove but rather that the claims be construed as encompassing all the features of patentable novelty in the present disclosure, including all features that would be treated as equivalents by persons having ordinary skill in the art of formulating topically-applied personal care, sunscreen, cosmetic, and dermatological products.

What is claimed is:

1. A composition that comprises an effective amount of one or more cosmetically suitable zinc oxide particles that reflects, refracts, or otherwise attenuates ultraviolet radiation; an effective amount of one or more cosmetically suitable titanium dioxide particles that reflects, refracts, or otherwise attenuates ultraviolet radiation; an effective amount of at least two cosmetically suitable siliceous compounds comprising an amorphous spherical silica composition and a composition comprising material derived from at least one diatomaceous algal species comprising a porous silica frustule structure; an effective amount of dimethicone and acrylates/dimethicone copolymer, bis-vinyl dimethicone/dimethicone copolymer, or a combination thereof; an amount of one or more cosmetically suitable waxes that is sufficient to promote the maintenance of a solid or semisolid structure at room temperature; and comprises no more than about 0.25% to about 5% water; wherein the composition exhibits an SPF of at least 30, has a critical wavelength of at least 370 nm, and is water resistant for at least about 80 minutes, and wherein the composition does not contain any detectable amount of an organic sunscreen ingredient.

2. The composition of claim 1, wherein the weight ratio of the zinc oxide particles to the titanium dioxide particles is about 1:4 to about 3:1.

3. The composition of claim 2, wherein most of the zinc oxide particles of the composition are mesoporous particles, and a detectable amount of the zinc oxide particles are contained in zinc oxide particle aggregates.

4. The composition of claim 3, wherein the weight ratio to the zinc oxide particles and titanium dioxide particles is about 1:55 to about 1:1.5.

5. The composition of claim 4, wherein the weight ratio of the diatomaceous algae composition to the amorphous spherical silica composition is about 1:20 to about 1:50.

6. The composition of claim 5, wherein the composition comprises no more than about 0.5% to about 5% water.

7. The composition of claim 6, wherein the diatomaceous algae composition comprises material derived from two distinct diatomaceous algal species.

8. The composition of claim 7, wherein the one or more cosmetically suitable waxes comprise a mineral wax, wherein the weight ratio of the mineral wax to the total of the one or more cosmetically suitable waxes is about 1:1.5 to 1:4.

9. The composition of claim 8, wherein the mineral wax is mostly composed of ozokerite.

10. The composition of claim 9, wherein the composition comprises no more than about 1% to about 5% water.

11. The composition of claim 10, wherein the composition further comprises an effective amount of at least one ingredient SPF booster compound, which significantly increases the SPF of the composition but does provide a significant amount of SPF protection in the absence of the zinc oxide particles and titanium dioxide particles.

12. The composition of claim 11, wherein the weight ratio of the SPF booster to the at least two siliceous compounds to the combination of the zinc oxide particles and titanium dioxide particles is about 20:3:20 to about 30:5:30.
